(19) United States
(12) Patent Application Publication
(10) Pub. No.: US 2024/0335651 A1
(43) Pub. Date: Oct. 10, 2024

(54) SEAL FOR A MECHANICAL CIRCULATORY SUPPORT DEVICE

(71) Applicant: KARDION GMBH, Stuttgart (DE)

(72) Inventors: Marvin Mitze, Stuttgart (DE); Vladimir Popov, Stuttgart (DE); Kenneth M. Martin, Woodside, CA (US); Hans Christof, Unterensingen (DE); Inga Schellenberg, Stuttgart (DE); Jens Burghaus, Stuttgart (DE); Tom Döhring, Esslingen (DE); Johannes Ferch, Esslingen (DE); Ingo Stotz, Ditzingen (DE); Johannes Bette, Stuttgart (DE); David Minzenmay, Stuttgart (DE)

(21) Appl. No.: 18/292,164

(22) PCT Filed: Aug. 2, 2022
(86) PCT No.: PCT/US2022/039209

§ 371 Date: Jan. 25, 2024

Related U.S. Application Data
(60) Provisional application No. 63/365,298, filed on May 25, 2022.
(60) Provisional application No. 63/229,436, filed on Aug. 4, 2021.

(51) Int. Cl. A61M 60/827; A61M 60/174; A61M 60/226; A61M 60/416; A61M 60/419

(52) U.S. Cl. CPC A61M 60/827; A61M 60/174; A61M 60/226; A61M 60/416; A61M 60/419

(57) ABSTRACT

The present disclosure is directed generally to mechanical cardiovascular support systems used in the medical field to assist the movement of blood. In particular the present disclosure is directed to mechanical cardiovascular support systems where an impeller is connected to a motor via a rotary drive shaft, the motor is contained in a motor compartment, the rotary drive shaft extends from the motor compartment, and a mechanical seal, for example a rotary shaft lip seal, prevents blood from entering the motor compartment. The seal may have an inverted radial shaft seal, have two opposing radial shaft seals, and/or have one or more elastomeric discs, among other features.

### BACKGROUND

The present disclosure is directed generally to devices deliverable to a patient's circulatory system, for example the left ventricle and aorta, to provide mechanical circulatory support. The present disclosure is directed more specifically to seals for mechanical circulatory support devices.

### SUMMARY

This disclosure is related to seals for mechanical circulatory support systems. Such systems may have an impeller rotated by a motor, and the seal mitigates or prevents blood flow from entering the compartment in which the motor is located. The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for mechanical circulatory support systems.

The following disclosure describes non-limiting examples of some embodiments of seals for mechanical circulatory support devices. For instance, other embodiments of the disclosed systems and methods may or may not include the features described herein. Moreover, disclosed advantages and benefits can apply only to certain embodiments and should not be used to limit the disclosure.

A first aspect of the disclosure is a seal for a heart pump, the seal comprising: a distal radial shaft seal configured to surround a shaft of the heart pump, with a flat side of the distal radial shaft seal facing distally and an open side of the distal radial shaft seal facing proximally; and a proximal radial shaft seal configured to surround the shaft and be located proximally of the distal radial shaft seal, such that the proximal radial shaft seal is located farther from an impeller of the pump than the distal radial shaft seal, with a flat side of the proximal radial shaft seal facing proximally and an open side of the proximal radial shaft seal facing distally.

A second aspect is the seal of aspect 1, wherein the distal radial shaft seal comprises a radially inner lip configured to contact the shaft and to extend from the flat side of the distal radial shaft seal in a proximal direction.

A third aspect is the seal of any of aspects 1 or 2, further comprising a distal spring located at least partially within the open side of the distal radial shaft seal and configured to compress a radially inner lip of the distal radial shaft seal radially inwardly onto the shaft.

A fourth aspect is the seal of any of aspects 1 to 3, further comprising a proximal spring located at least partially within the open side of the proximal radial shaft seal and configured to compress a radially inner lip of the proximal radial shaft seal radially inwardly onto the shaft.

A fifth aspect is the seal of any of aspects 1 to 4, further comprising one or more discs comprising a central opening with an inner diameter configured to be less than the outer diameter of the shaft.

A sixth aspect is the seal of aspect 5, wherein a radially inner edge of the central opening of each of the discs is configured to wear off in response to rotation of the shaft.

A seventh aspect is the seal of any of aspects 1 to 6, further comprising grease located between the distal radial shaft seal and the middle disc and between the middle disc and the proximal radial shaft seal.

An eighth aspect is the seal of any of aspects 1 to 7, wherein each of the distal and proximal radial shaft seals have radially outer lips configured to contact an inner side of a housing.

A ninth aspect is the seal of any of aspects 1 to 1, wherein the seal is configured to be assembled with the heart pump and delivered to the heart via a catheter.

A tenth aspect is the seal of any of aspects 1 to 8, further comprising a housing having a distal end wall and a cylindrical side wall extending proximally from the distal end wall, the distal end wall having a distal side configured to contact blood flow and having a central opening configured to receive therethrough the shaft, wherein the distal radial shaft seal is configured to be located proximally of the distal end wall at least partially within the housing.

Another aspect is a heart pump (22) comprising a motor (145) having a rotor; an impeller (72) for providing a blood flow; a drive shaft (140) that is connected to the rotor and the impeller; and a seal element (156) that is disposed between the motor and the impeller, wherein the seal element (156) includes a central aperture for receiving the drive shaft (140) in sliding sealing contact, such that the motor (145) is sealed from the blood flow.

Another aspect is a heart pump that uses a barrier fluid to prevent blood from entering the motor of the heart pump. Thus, the operating time of the heart pump can be extended. A corresponding heart pump comprises a housing, an impeller, a motor, a scaling element and a barrier fluid. The housing has an interior and an opening to the interior. The impeller has at least one blade, the impeller being positioned next to the opening. The motor is located in the interior of the housing and has a shaft which passes through the opening and is coupled to the impeller to drive the impeller. The sealing element is located between the impeller and the motor housing and is designed to seal a gap between the impeller and the housing. The barrier fluid is located between the sealing element and the shaft, which are arranged and designed to prevent a medium from entering the interior of the motor from an environment surrounding the heart pump. The impeller is driven by the motor via the shaft. The sealing element can be ring-shaped. The sealing element can be attached to the impeller. This allows the sealing element to rotate with the impeller. Alternatively, the sealing element can be attached to the motor housing. Regardless of the mounting, a gap between the impeller and the motor housing can be sealed using the seal element. The barrier fluid can also be located inside the motor. In this case, there is no need for an additional sealing element to prevent the barrier fluid from entering the interior through the opening. According to a design form, the sealing element can be designed as a contact or non-contact seal. Thus, any suitable sealing form can be used. Furthermore, the sealing element can be designed as a labyrinth seal and additionally or alternatively as a gap seal. Such seals are wear-free and have low friction. In addition, the heart pump may have a second sealing element. The second sealing element may be located at the opening and may be designed to seal the interior of the motor housing against a space between the motor housing and the impeller. The barrier fluid may be located in the gap. In this way, the barrier fluid can be retained from entering the motor interior. Furthermore, the heart pump may have at least one bearing, the bearing being designed to support the shaft inside the housing. Advantageously, the shaft can also be centered by the at least one bearing. The barrier fluid can be a biocompatible medium. This means that the barrier fluid has no negative influence on the patient in the event of a leakage of the heart pump. According to one design, the barrier fluid can consist of glucose and/or endogenous fat. This ensures optimal biocompatibility.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a cross sectional view of a distal end of an embodiment of a mechanical circulatory support (MCS) system supported by a catheter and positioned across an aortic valve.

FIG. 2 schematically illustrates an embodiment of an MCS system inserted into the body via the access pathway from the femoral artery to the left ventricle.

FIG. 3 is a side elevational view of an embodiment of an MCS system that may incorporate the various features described herein.

FIG. 4 is the system of FIG. 3, shown with the introducer sheath removed and including an insertion tool and a guidewire back loading aid.

FIG. 5 is a side view of an introducer kit, having a sheath and dilator, and that may be used with the various MCS systems and methods described herein.

FIG. 6 shows a placement guidewire that may be used with the various MCS systems and methods described herein.

FIG. 7 is a perspective fragmentary view of a distal pump region of the MCS system of FIG. 1.

FIG. 8 is a side elevational view of a distal region of the MCS system of FIG. 1, showing the guidewire path and the guidewire back loading aid in place.

FIG. 9A is a side view of one embodiment of an MCS device that may be used with the MCS system of FIG. 1.

FIG. 9B is a partial cross-sectional view of the MCS device of FIG. 9A showing an embodiment of a seal.

FIG. 10 is a partial cross-sectional view of another embodiment of an MCS device having a distal facing lip seal and a distal protection disc.

FIG. 11 is a partial cross-sectional view of another embodiment of an MCS device having a distal facing lip seal, a distal protection disc, and a proximal disc.

FIG. 12 is a partial cross-sectional view of another embodiment of an MCS device having a proximal facing lip seal and a proximal disc.

FIG. 13A is a partial cross-sectional view of another embodiment of an MCS device having a distal facing lip seal and a distal protection disc having a contoured face and an impeller with a matching contour.

FIG. 13B is a partial cross-sectional view of another embodiment of an MCS device having a distal facing lip seal and a distal protection disc having a contoured face and an impeller with a non-matching contour (e.g., flat).

FIG. 14A is a partial cross-sectional view of another embodiment of an MCS device having two lip seals facing one another, optionally with one garter spring or two.

FIG. 14B is a partial cross-sectional view of another embodiment of an MCS device having two lip seals facing one another, showing an optional leading edge on the distal lip seal.

FIG. 14C is a partial cross-sectional view of another embodiment of an MCS device having two lip seals facing one another, showing an optional leading edge on the distal protection disc.

FIG. 15 is a partial cross-sectional view of another embodiment of an MCS device having a pressure balancing lubricant reservoir.

FIG. 16A is a partial cross-sectional view of another embodiment of an MCS device having two lip seals facing one another, a distal disc, a middle disc, and a proximal disc contained in a seal housing.

FIG. 16B is an isometric, exploded, partially cut-away view of the seal components of FIG. 16A.

FIG. 16C is a cross-sectional view of the seal components of FIG. 16A shown isolated as a subassembly for facilitating manufacturing and assembly.

FIG. 16D is a side cross-sectional view of another embodiment of a seal assembly, where a proximal disc has an extended radial contact surface and an axial contact surface.

FIG. 16E is a perspective cross-sectional view of an embodiment of a seal assembly and impeller, where the seal assembly has a distally tapered distal seal container.

FIGS. 16F and 16G are various views of a seal assembly, an impeller and a flow channel with a transparent housing for clarity, where the seal assembly has a distally tapered distal seal container and outlet strut support members.

FIG. 17A is an isometric illustration of an embodiment of an impeller with a smooth base surface.

FIGS. 17B and 17C are isometric illustrations of embodiments of impellers having proximal vanes, in contrast to the impeller of FIG. 17A, which may optionally be used with any MCS devices or seals described herein, for example those shown in FIG. 9B, 10, 11, 12, 14A, 14B, 14C, 15, 16A, 16B, or 16C.

FIG. 18A is a cross-sectional view of an embodiment of an impeller fastened to a drive shaft via an impeller base plate that may be used with the various MCS systems described herein.

FIG. 18B is a cross-sectional view of an embodiment of an impeller fastened directly to a drive shaft that may be used with the various MCS systems described herein.

FIG. 18C is an isometric cut-away view of an embodiment of an impeller fastened to a drive shaft with a locking key that may be used with the various MCS systems described herein.

FIG. 19 is a cross-sectional view of another embodiment of an MCS device having an axial lip seal and a radial face lip seal that may be used with the various MCS systems described herein.

### DETAILED DESCRIPTION

The disclosure herein is related to a mechanical circulatory support (MCS) system and device having an impeller connected to a drive shaft that is driven by a motor, wherein blood is prevented from entering the motor with one or more seals and/or other barrier features. The following detailed description is directed to certain specific embodiments. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings.

### A. Mechanical Circulatory Support (MCS) System

The sealing components described herein may be part of a Mechanical Circulatory Support (MCS) device or MCS system 10 such as the system in the following description.

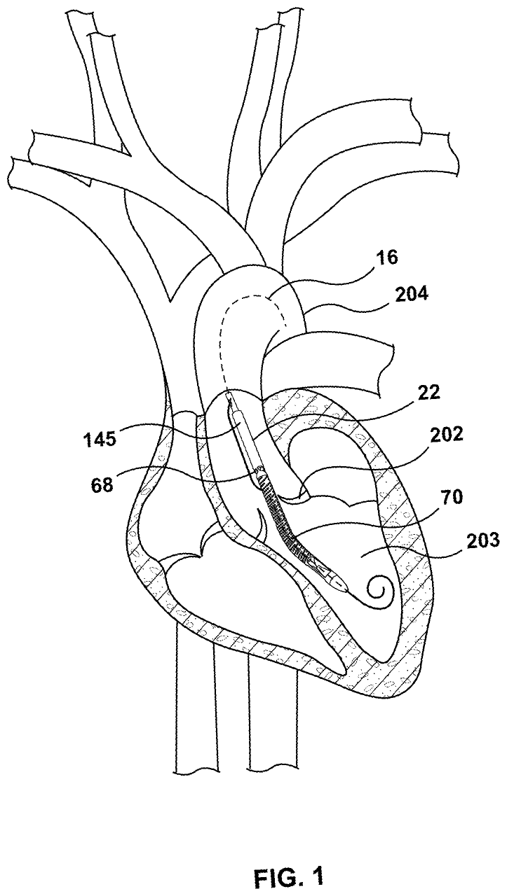

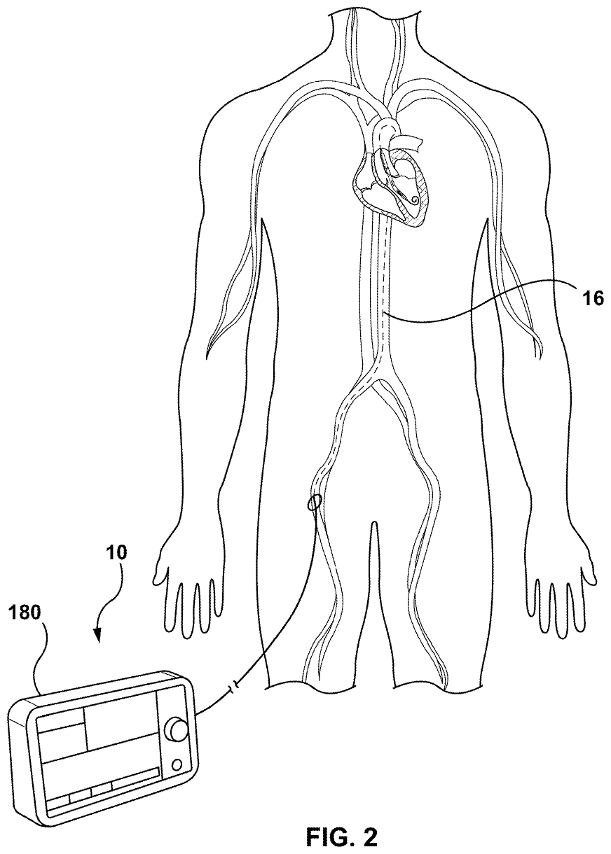

As shown in FIGS. 1 and 2, the MCS system 10 may include a temporary (generally no more than about 6 hours, or no more than about 3 hours, 4 hours, 5 hours, 7 hours, 8 hours, or 9 hours) left ventricular support pump 22 for use during various procedures, such as high-risk percutaneous coronary intervention (PCI) performed in elective or urgent, hemodynamically stable patients with severe coronary artery disease and/or depressed left ventricular ejection fraction. The system 10 may be used if a heart team, including a cardiac surgeon, has determined high risk PCI is the appropriate therapeutic option. It is placed across the aortic valve, for example via a single femoral arterial access.

The MCS system 10 includes a low-profile axial rotary blood pump mounted on a catheter such as an 8 French (Fr) catheter 16, where 1 Fr equals ⅓ millimeter (mm). The pump may be referred to as an MCS pump or MCS device. When in place, the MCS pump can be driven by an MCS controller 180 to provide up to about 4.0 liters/minute of partial left ventricular support, at about 60 mm Hg. No system purging is needed due to scaled motor. An improved bearing design can also avoid the need for purging. By “purging” it is meant that the system need not have a glucose or other type liquid purge repeatedly introduced into the system through tubing in order to prevent contamination of the motor by the blood. The MCS system 10 thus avoids the complexity associated with systems that need purging, and results in a simpler, less expensive device that is easier to use. The system may be visualized fluoroscopically, eliminating the need for placement using sensors.

The system may further include an expandable sheath 12. The sheath 12 may allow 8-10 Fr initial access size for easy insertion and closing, expandable to allow introduction of 14 Fr and 18 Fr pump devices, and return to a narrower diameter around the 8 Fr catheter once the pump has passed. This feature may allow passage of the heart pump through vasculature while minimizing shear force within the blood vessel, advantageously reducing risk of bleeding and healing complications. Distention or stretching of an arteriotomy may be done with radial stretching with minimal shear, which is less harmful to the vessel. Access may be accomplished via transfemoral, transaxillary, transaortal, or transapical approach.

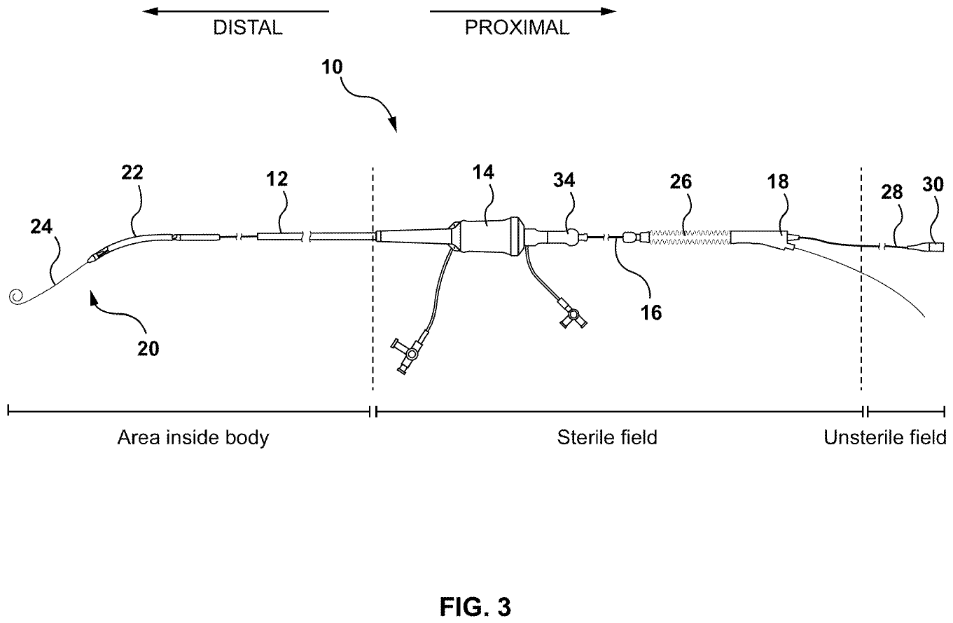

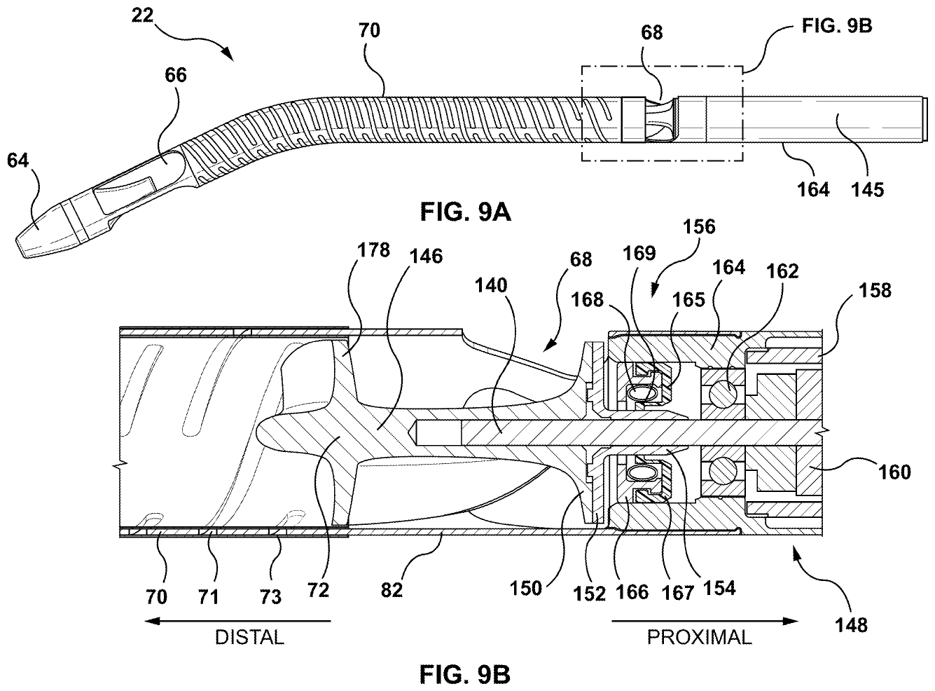

FIG. 1 further shows a distal end of the MCS system 10 having the pump 22 mounted on the tip of an 8 Fr catheter 16. As used herein, “distal” and “proximal” refer to directions along the MCS system 10 in use that are, respectively, farther from and closer to the body, as further shown in FIGS. 3 and 9B as examples. An inlet tube portion 70 of the device extends across the aortic valve 202. An impeller is located at the outflow section 68 of the inlet tube, drawing blood from the left ventricle 203 through the inlet tube portion 70 and ejecting it out the outflow section 68 into the ascending aorta 204. The motor 145 is mounted proximal, which may be directly proximal, to the impeller in a sealed housing eliminating the need to flush the motor prior to or during use. This configuration provides hemodynamic support during high-risk PCI, time and safety for a complete revascularization via a minimally invasive approach (rather than an open surgical procedure).

The system has been designed to eliminate the need for motor flushing. The system also provides increased flow performance up to 4.0 l/min at 60 mmHg with acceptably safe hemolysis due to a computational fluid dynamics (CFD) optimized impeller that minimizes shear stress. The seal and other features as described herein contribute to these and other advantages.

The MCS device 10 actively unloads the left ventricle by pumping blood from the ventricle into the ascending aorta and systemic circulation (shown in FIGS. 1 and 2). When in place, the MCS device can be driven by the complementary MCS Controller to provide between 0.4 liters per minute (l/min) up to 4.0 l/min of partial left ventricular support.

In general, the overall MCS system 10 may include a series of related subsystems and accessories, including one or more of the following:
The MCS device 10 may include a pump, shaft, proximal hub, insertion tool, proximal cable, infection shield and guidewire aid. The MCS Device may be provided sterile;
The MCS shaft may contain the electrical cables and a guidewire lumen for over-the-wire insertion;
The proximal hub may contain guidewire outlet with a valve to maintain hemostasis and connect the MCS shaft to the proximal cable, that connects the MCS Device to the MCS Controller;
The proximal cable may be 3.5 meters (m) (approximately 177 inches (in)) in length and extend from the sterile field to the non-sterile field where the MCS Controller is located;
An MCS insertion tool may be part of the MCS device 10 to facilitate the insertion of the pump into an introducer sheath and to protect the inlet tube and the valves from potential damage or interference when passing through the introducer sheath;
A peel-away guidewire aid may be pre-mounted on the MCS device 10 to facilitate the insertion of the 0.018″ placement guidewire into the pump and into the MCS shaft;
A 3 meter long, 0.018″ wide placement guidewire may be used, having a soft coiled pre-shaped tip for atraumatic wire placement into the left ventricle. The guidewire may be provided sterile;
A 14 Fr introducer sheath with a usable length of 275 mm may be used to maintain access into the femoral artery and provide hemostasis for a 0.035″ guidewire, the diagnostic catheters, the 0.018″ placement guidewire, and the insertion tool. The housing of the introducer sheath may be designed to accommodate the MCS insertion tool. The introducer sheath may be provided sterile;
An introducer dilator may be compatible with the introducer sheath to facilitate atraumatic insertion of the introducer sheath into the femoral artery. The introducer dilator may be provided sterile; and/or
An MCS controller may drive and/or operate the MCS device, observe its performance and condition as well as provide error and status information. The powered controller may be designed to support at least about 12 hours of continuous operation and contain a basic interface to indicate and adjust the level of support provided to the patient. Moreover, the controller may provide an optical and audible alarm notification in case the system detects an error during operation. The MCS Controller may be provided non-sterile and be contained in an enclosure designed for cleaning and re-use outside of the sterile field. The controller enclosure may contain a socket into which the extension cable is plugged.

Referring to FIG. 3, there is illustrated an overall MCS system 10 in accordance with one aspect of the present development, subcomponents of which will be described in greater detail below. The system 10 includes an introducer sheath 12 having a proximal introducer hub 14 with a central lumen for axially movably receiving an MCS shaft 16. The MCS shaft 16 extends between a proximal hub 18 and a distal end 20. The hub 18 may be provided with an integrated microcontroller or memory storage device for device identification and tracking of the running time, which could be used to prevent overuse to avoid excessive wear or other technical malfunction. The microcontroller or memory device could disable the device, for example to prevent using a used device. They could communicate with the controller, which could display information about the device or messages about its usage. An atraumatic cannula tip with radiopaque material allows the implantation/explanation to be visible under fluoroscopy.

A pump 22 is carried by a distal region of the MCS shaft 16. The system 10 is provided with at least one central lumen for axially movably receiving a guide wire 24. The proximal hub 18 is additionally provided with an infection shield 26. A proximal cable 28 extends between the proximal hub 18 and a connector 30 for releasable connection to a control system typically outside of the sterile field, to drive the pump 22. The pump 22 may include any of the seal embodiments described herein, such as those described with and shown in FIG. 9B-16C, 18A, 18B or 19.

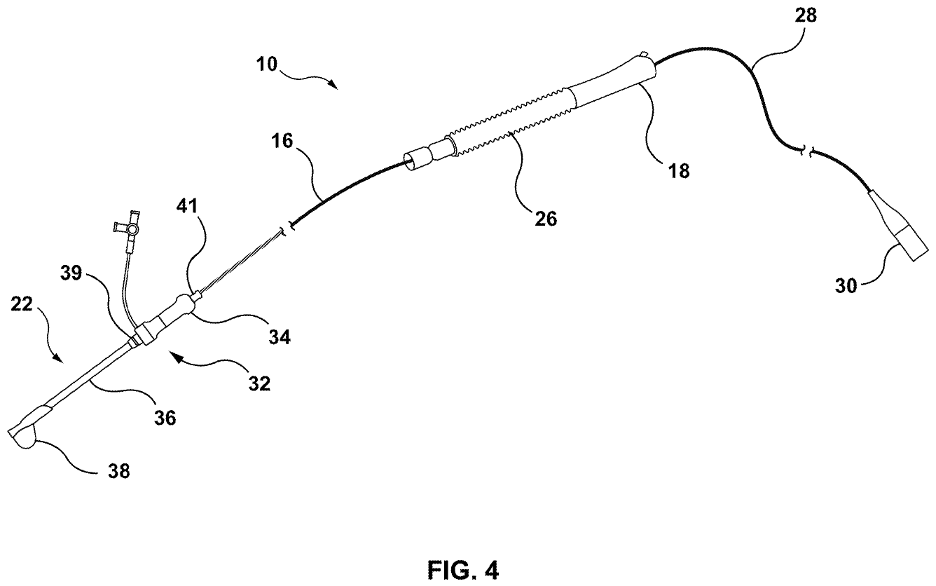

Referring to FIG. 4, the system 10 additionally includes an insertion tool 32, having an elongate tubular body 36 having a length within the range of from about 85 mm to about 160 mm (e.g., about 114 mm) and an inside diameter within the range of from about 4.5 mm to about 6.5 mm (e.g., about 5.55 mm), extending distally from a proximal hub 34. The tubular body 36 includes a central lumen adapted to axially movably receive the MCS shaft 16 and pump 22 there through, and sufficient collapse resistance to maintain patency when passed through the hemostatic valves of the introducer sheath. As illustrated in FIG. 4, the pump 22 can be positioned within the tubular body 36, such as to facilitate passage of the pump 22 through the hemostatic valve(s) on the proximal end of an introducer hub 14. A marker 37 (FIG. 7) is provided on the shaft 16 spaced proximally from the distal tip 64 such that as long as the marker 37 is visible on the proximal side of the hub 34, the clinician knows that the pump is within the tubular body 36.

The hub 34 may be provided with a first engagement structure 39 for engaging a complimentary second engagement structure on the introducer sheath to lock the insertion tool into the introducer sheath. The hub 34 may also be provided with a locking mechanism 41 for clamping onto the shaft 16 to prevent the shaft 16 from sliding proximally or distally through the insertion tool once the MCS device has been positioned at the desired location in the heart. The hub 34 may additionally be provided with a hemostasis valve to seal around the shaft 16 and also accommodate passage of the larger diameter MCS device which includes the pump. In one commercial presentation of the system, the MCS device as packaged is pre-positioned within the insertion tool and the guidewire aid is pre-loaded within the MCS device and shaft 16, as illustrated in FIG. 4.

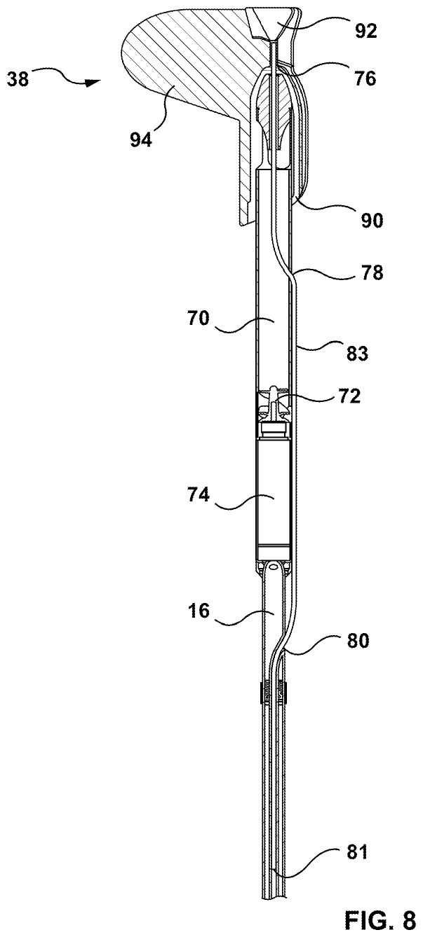

A guidewire aid 38 (also illustrated in FIG. 8) includes a proximal opening 90 configured to slip over and removably receive the distal tip 64 and/or struts at the distal end of the inlet tube 70 that define windows of the pump inlet 66. A guidewire guide tube 83 having a lumen therethrough is positioned within the proximal opening 90 and aligned to pass through the guidewire port 76 of the distal tip 64. The lumen of the guidewire guide tube 83 is in communication with a distal flared funnel opening 92 which gets larger in cross-section in the distal direction. The guidewire aid 38 may be provided assembled on the MCS pump 22 with the guidewire guide tube 83 pre-loaded along a guidewire path, for example into the MCS pump 22 through port 76, through a portion of the fluid path within the inlet tube 70, out of the MCS pump 22 through port 78, along the exterior of the MCS pump and into the shaft 16 through port 80. This helps a user guide the proximal end of a guide wire into the funnel 92 through the guidewire path and into the guidewire lumen of the MCS shaft 16. A pull tab 94 may be provided on the guide wire aid 38 to facilitate grasping and removing the guidewire aid, including the guidewire guide tube 83, following loading of the guidewire. The guidewire aid 38 may have a longitudinal slit or tear line, for example along the funnel 92, proximal opening 90 and guidewire guide tube 83, to facilitate removal of the guidewire aid 38 from the MCS pump 22 and guidewire 100.

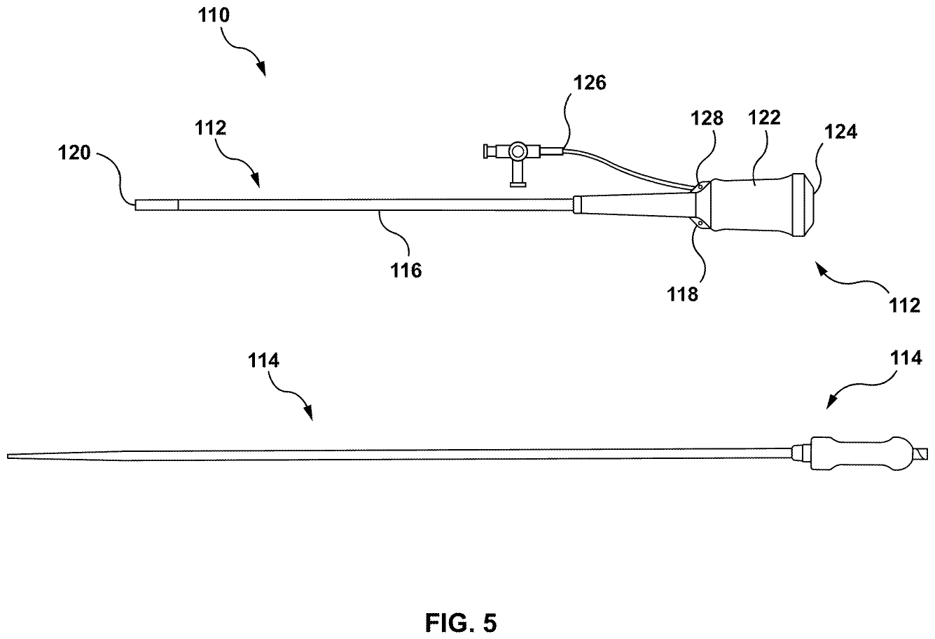

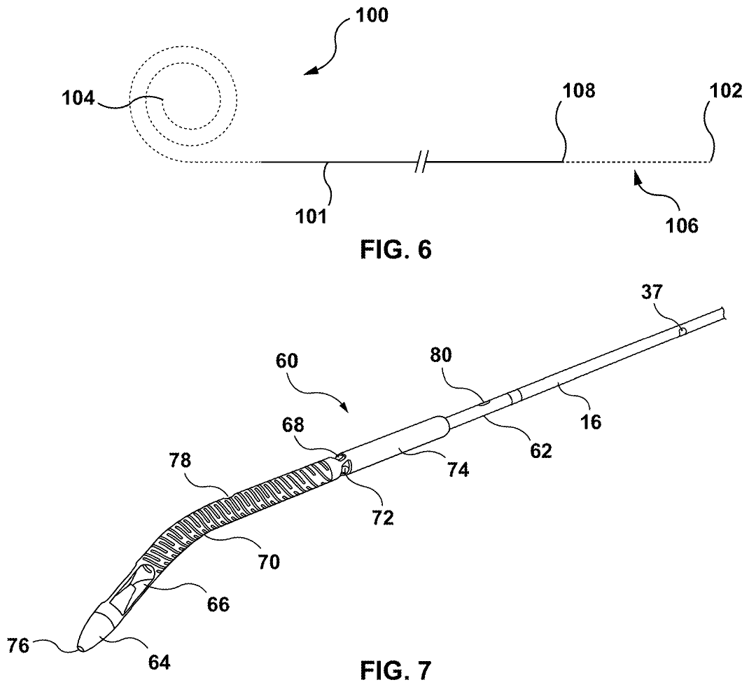

Referring to FIGS. 5 and 6, an introducer kit 110 may include a guidewire 100, an introducer sheath 112, a dilator 114, and/or a guidewire aid 38, for example as discussed above. The guidewire 100 comprises an elongate flexible body 101 extending between a proximal end 102 and a distal end 104. A distal zone of the body 101 may be pre-shaped into a J tip or a pigtail, as illustrated in FIG. 6, to provide an atraumatic distal tip. A proximal zone 106 is configured to facilitate threading through the MCS device and extends between the proximal end 102 and a transition 108. The proximal zone 106 has an axial length within the range of from about 100 mm to about 500 mm (e.g., about 300 mm).

The introducer kit 110 comprises a sheath 112 and a dilator 114. The sheath 112 comprises an elongate tubular body 116, extending between a proximal end 118 and a distal end 120. The tubular body 116 terminates proximally in a proximal hub 122. Optionally, the tubular body 116 is expandable or can be peeled apart. The proximal hub 122 includes a proximal end port 124 in communication with a central lumen extending throughout the length of the tubular body 116 and out through a distal opening, configured for axially removably receiving the elongate dilator 114. Proximal hub 122 is additionally provided with a side port 126, at least one and optionally two or more attachment features such as an eye 128 to facilitate suturing to the patient, and at least one and optionally a plurality of hemostasis valves for providing a seal around a variety of introduced components such as a standard 0.035″ guidewire, a 5 Fr or 6 Fr diagnostic catheter, an 0.018″ placement guidewire 100, and the insertion tool 32.

Additional details of the distal, pump region of the MCS system are illustrated in FIG. 7. Pump zone 60 extends between a bend relief 62 at the distal end of shaft 16 and a distal tip 64. A pump inlet 66 is in fluid communication with a pump outlet 68 by way of a flow path extending axially through the inlet tube 70. The pump inlet may be positioned at about the transition between the inlet tube and the proximal end of distal tip 64, which may be generally within about 5 cm or less or 3 cm or less from the distal port 76. In some embodiments, the distal tip 64 is radiopaque. For example, the distal tip 64 may be made from a polymer containing a radiopacifier such as barium sulfate, bismuth, tungsten, iodine. In some embodiments, an entirety of the MCS device is radiopaque. In some embodiments, a radiopaque marker is positioned on the inlet tube between the pump outlet 68 and the guidewire port 78 to indicate the current position of the aortic valve. Inlet tube 70 may comprise a highly flexible slotted (e.g., laser cut) metal (e.g., Nitinol) tube having a polymeric (e.g., Polyurethane) tubular layer to isolate the flow path. Inlet tube may have an axial length within the range of from about 60 mm and about 100 mm and in one implementation is about 67.5 mm. The outside diameter is typically within the range of from about 4 mm to about 5.4 mm, and in one implementation is about 4.66 mm. The connections between the inlet tube and the distal tip and to the motor may be secured such as through the use of laser welding, adhesives, threaded or other interference fit engagement structures, or may be via press fit.

The impeller 72 is positioned in the flow path between the pump inlet 66 and pump outlet 68. In the illustrated embodiment, the impeller 72 is positioned adjacent to the pump outlet 68. As is discussed further below, the impeller 72 is rotationally driven by a motor contained within motor housing 74, on the proximal side of the impeller 72.

The MCS device can be provided in either a rapid exchange or over the wire configuration. A first guide wire port 76 is in communication, via a first guide wire lumen through the distal tip component 64 and at least a portion of the flow path in the inlet tube, with second guide wire port 78 extending through a side wall of the inlet tube 70, and distal to the impeller 72. This could be used for rapid exchange, with the guidewire extending proximally alongside the catheter from the second guidewire port 78.

The catheter may be provided in an over-the-wire configuration, in which the guidewire extends proximally throughout the length of the catheter through a guidewire lumen. In the over the wire embodiment of FIG. 7, however, the guidewire exits the catheter via second guidewire port 78, extends proximally across the outside of the impeller and motor housing, and reenters the catheter shaft 16 via third guidewire port 80. See also FIG. 8. The third guide wire port 80 is located proximal to the motor, and, in the illustrated embodiment, is located on the bend relief 62. Third guide wire port 80 is in communication with a guide wire lumen which extends proximally throughout the length of the shaft 16 and exits at a proximal guidewire port carried by the proximal hub 18.

The pump may be provided assembled with a removable guidewire aid 38 having a guidewire guide tube 83 which tracks the intended path of the guidewire from the first guidewire port 76, proximally through the tip 64 and outside of the inlet tube via second guide wire port 78 and into the catheter via third guidewire port 80. In the illustrated implementation, the guidewire guide tube extends proximally within the catheter to a proximal end 81, in communication with, or within the guidewire lumen which extends to the proximal hub 18. The guidewire guide proximal end 81 may be positioned within about 5 mm or 10 mm of the distal end of the shaft 16, or may extend into the catheter shaft guidewire lumen for at least about 10 mm or 20 mm, such as within the range of from about 10 mm to about 50 mm. The proximal end of a guidewire 102 may be inserted into the funnel 92, passing through the first (distal) guidewire port 76 and guided along the intended path by tracking inside of the guidewire guide tube. The guidewire guide tube may then be removed, leaving the guidewire in place.

In one implementation, the distal end of the guidewire guide tube 83 is attached to the pull tab 94 of guide wire aid 38 and provided with an axially extending split line such as a weakening, slot or perforated tearable line. Removal may be accomplished such as by grasping the pull tab 94 and pulling out the guide wire tube as it splits and peels away along the split line. The inside surface of guide tube 83 may be provided with a lubricious coating, such as polytetrafluoroethylene (PTFE).

FIG. 9A is a side view of one embodiment of an MCS device that may be used with the MCS system 10. FIG. 9B is a partial cross-sectional view of a region of the MCS device as indicated in FIG. 9A, showing an embodiment of a seal, among other features. Referring to FIGS. 9A and 9B, the impeller 72 is attached to a rigid motor drive shaft 140, which may be relatively short (e.g., in a range of 29 mm to 34 mm). Some of the features disclosed herein such as sealing elements (159) may be adapted for use with a heart pump having a motor that is kept external to the body and connected to an impeller that is in the heart with a long flexible driveshaft, which may have a length in a range of 1200 mm to 1500 mm. In the illustrated implementation, the drive shaft 140 extends distally into a proximally facing central lumen 142 in the impeller 72, such as through a proximal extension 154 on the impeller hub 146, where it may be secured by a press fit, laser weld, adhesives or other bonding technique. The impeller 72 includes a radially outwardly extending helical blade 178, which, at its maximum outside diameter, is spaced apart from the inside surface of tubular impeller housing 82. The blade 178 may be spaced within the range of from about 40 μm to about 120 μm. Impeller housing 82 may be a proximal extension of the inlet tube 70, on the proximal side of the slots 71 formed in the inlet tube 70 to provide flexibility distal to the impeller. A tubular outer membrane 73 encloses the inlet tube and seals the slots 71 while preserving flexibility of the inlet tube. Pump outlets 68 are formed in the sidewall of the impeller housing, axially aligned for example with a proximal portion of the impeller (e.g., a proximal 25% to 50% portion of the impeller).

The impeller 72 may comprise a medical grade titanium. This enables a computational fluid dynamics (CFD) optimized impeller design with minimized shear stress for reduced damage of the blood cells (hemolysis) and a non-constant slope increasing the efficiency. This latter feature cannot be accomplished with a mold-based production method. Electro polishing of the surface decreases the surface roughness to minimize the impact on hemolysis.

In some embodiments, the impeller hub 146 flares radially outwardly in a proximal direction to form an impeller base 150, which may direct blood flow out of the outlets 68. A proximal surface of the impeller base 150 is secured to an impeller base plate 152, which may be in the form of a radially outwardly extending flange, secured to the motor shaft 140. For this purpose, the impeller base plate 152 may be provided with a central aperture to receive the motor shaft 140 and may be integrally formed with or bonded to a tubular sleeve 154 adapted to be bonded to the motor shaft 140. In one implementation, the impeller base plate 152 is first attached to the motor shaft 140 and bonded such as through the use of an adhesive. In a second step, the impeller 72 may be advanced over the shaft and the impeller base 150 bonded to the impeller base plate 152 such as by laser welding.

The distal opening in the aperture in impeller base plate 152 may increase in diameter in a distal direction, to facilitate application of an adhesive. The proximal end of tubular sleeve 154 may decrease in outer diameter in a proximal direction to form an entrance ramp for facilitating advancing the sleeve proximally over the motor shaft and through the motor seal 156, discussed further below.

The pump includes a motor 145 sealed from the blood flow, due to the short time of usage for high risk PCI (in some embodiments, no more than about 6 hours), configured for use without flushing or purging. This provides the opportunity to directly bond the impeller 72 on the motor shaft 140 as discussed in further detail below, removing issues sometimes associated with magnetic coupling such as the additional stiff length, space requirements or pump efficiency.

Motor 145 includes a stator 158 having conductive windings surrounding a cavity which encloses motor armature (rotor) 160 which may include a plurality of magnets rotationally secured with respect to motor shaft 140. The motor shaft 140 extends from the motor 145 through a rotational bearing 162 and also through a seal 156 before exiting the sealed motor housing 164.

The seal 156 includes a seal holder 166 which supports an annular seal ring 167, such as a polymeric seal ring. The seal ring 167 includes a central aperture for receiving the sleeve 154, or alternatively the drive shaft 140, and is biased radially inwardly against the sleeve 154 to maintain the seal ring in sliding sealing contact with the rotatable sleeve 154. A spring 168, for example a garter spring made which may be made from spring-stainless steel or superelastic Nitinol, fits in a groove between the annular seal 167 and the seal holder 166 and applies an inward facing force against the flexible annular seal 167, which in turn maintains a contact force between a lip 169 of the annular seal 167 and the rotating shaft 140 or sleeve 154 within the central aperture. The outside surface of the sleeve 154 or drive shaft 140 may be provided with a smooth surface such as by electro polishing, to minimize wear on the seal. The outside surface of the sleeve 154 or drive shaft 140 may be provided with a surface treatment or coating such as a hydrophobic or hydrophobic treatment such as an applied coating or a micropatterned surface, to minimize wear on the seal.

As shown in FIG. 9B, the orientation of the seal may include having the annular seal 167 proximal to the seal holder 166 with the seal holder facing distally toward the impeller 72, wherein the distal face of the seal holder 166 is in contact with flowing blood. The seal 156 prevents blood from passing the annular seal into the motor housing, which is proximal to the seal.

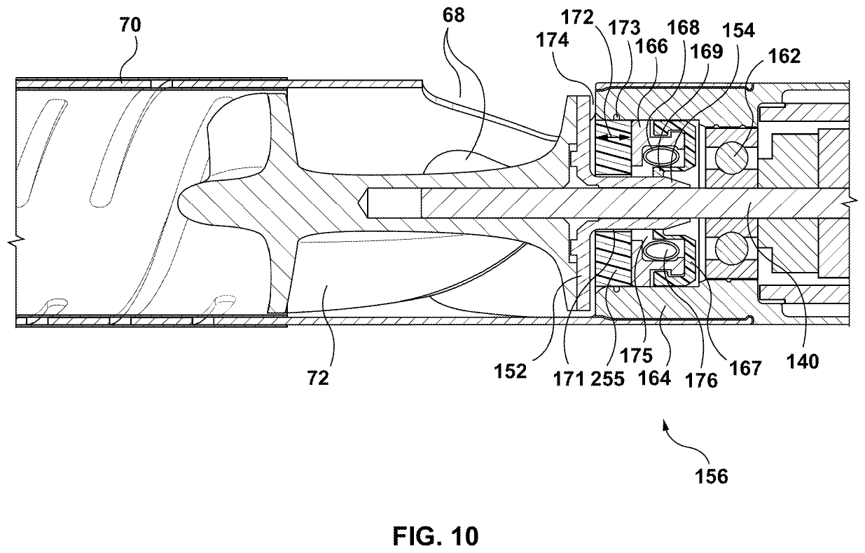

This is merely one example of a seal that may be used with the MCS device and pump 22. Other embodiments of seals that may be used on the various MCS devices are described herein, for example with respect to FIGS. 10-16C, 18A, 18B and 19.

Further, the seal, vane and other features described herein may be used with a variety of different MCS systems and devices, and vice versa. For example, any of the seal, vane and/or other features described herein may be used with any of the features, for example the MCS system and device features as described in U.S. provisional application No. 63/116,616, filed Nov. 20, 2020 and titled Mechanical Left Ventricular Support System for Cardiogenic Shock, in U.S. provisional application No. 63/116,686, filed Nov. 20, 2020 and titled Mechanical Circulatory Support System for High Risk Coronary Interventions, in U.S. provisional application No. 63/224,326, filed Jul. 21, 2021 and titled Guidewire, in international PCT applications no. PCT/EP2019/076002 filed Sep. 26, 2019 and titled Scaled Micropump, in PCT/EP2019/062731 filed May 16, 2019 and titled Permanent-magnetic radial rotating joint and micropump comprising such a radial rotating joint, in PCT/EP2019/062746 filed May 16, 2019 and titled Rotor bearing system, in PCT/EP2019/064775 filed Jun. 6, 2019 and titled Line device for a ventricular assist device and method for producing a line device, in PCT/EP2019/064780 filed Jun. 6, 2019 and titled Sensor head device for a minimal invasive ventricular assist device and method for producing such a sensor head device, in PCT/EP2019/064136 filed May 30, 2019 and titled Line device for conducting a blood flow for a heart support system, and production and assembly method, in PCT/EP2019/064807 filed Jun. 6, 2019 and titled Method for determining a flow speed of a fluid flowing through an implanted, vascular assistance system and implantable, vascular assistance system, in PCT/EP2019/071245 filed Aug. 7, 2019 and titled Device and method for monitoring the state of health of a patient, in PCT/EP2019/071233 filed Aug. 7, 2019 and titled Bearing device for a heart support system, and method for rinsing a space in a bearing device for a heart support system, in PCT/EP2019/068434 filed Jul. 9, 2019 and titled Impeller housing for an implantable, vascular support system, in PCT/EP2019/069571 filed Jul. 19, 2019 and titled Feed line for a pump unit of a cardiac assistance system, cardiac assistance system and method for producing a feed line for a pump unit of a cardiac assistance system, and/or in PCT/EP2019/075662 filed Sep. 24, 2019 and titled Method and system for determining a flow speed of a fluid flowing through an implanted, vascular assistance system; the entire disclosure of each of which is incorporated by reference herein for all purposes and forms a part of this specification and description.

### B. Control of Motor Speed with a Device Having a Rotary Shaft Seal

The controller 180 may be adapted to provide power to the motor 145 to maintain a target motor speed even when the current draw changes. The rotational speed of the impeller is directly related to the rotational speed of the driveshaft since they are rigidly connected. The flow rate of blood moved by the impeller is a function of the rotational speed of the impeller. The lip of a rotary shaft seal or the contacting surface of elastomeric fluid barriers may be designed to wear down during the duration of use. Frictional force applied to the rotary shaft by these parts may be expected to decrease over time as the part wears down. Motor current draw in a brushless DC motor, which is a function of the frictional force and other factors such as pressure differential, may likewise decrease over time in response to decreased friction. One way for a controller to detect motor speed may include the use of hall sensors in the MCS pump 22 or another portion of the device, which provide a signal to the controller that is an indication of rotational speed. Alternatively, a field-oriented control (FOC) motor may be used, which beneficially allows an MCS device to be smaller by excluding the need for extra sensors. Smaller sized MCS devices, (e.g., <18 FR, <16 FR, <14 FR, about 14 Fr) may be particularly advantageous for high-risk PCI procedures. An FOC motor maybe used to measure the back-EMF that occurs while the motor is spinning. This back EMF has a rhythmic characteristic (e.g., sinusoidal) that represents the frequency of the motor's rotation, which may be detected by the controller as a feedback signal in a control algorithm, which may include a form of PID (proportional-integral-derivative) control. The controller may adjust current delivery to the motor according to the feedback signal to adjust the motor speed so it matches the target speed, which may include allowance for small fluctuations around the target, for example, fluctuation of plus or minus 0.006% of the target motor speed (e.g., about 250 rpm for a target speed of 40k rmp) may be allowed without the controller making adjustments to the motor current.

### C. Seal Configuration and Principle of Sealing

Without being bound by theory, rotary shaft lip seals are typically oriented with the lip facing the higher-pressure side, that is to say, the side that has a fluid that the seal is meant to prevent from passing to the other side. For example, the seals shown in FIGS. 9B, 10, 11, 13A and 13B are shown oriented this way. As shown in FIG. 9B for example, the seal 167 has a radially inner lip 169 extending distally from a proximal side 165 of the seal 167. The open distal side of the seal 167 having the cavity faces distally toward the fluid side and impeller 72, and the opposite proximal side 165, which may be flat, faces proximally in the opposite direction toward the motor 145.

Conventional sealing arrangements may have the seal 167 with the open side facing distally, as described, as well as have the inner lip 169 contacting the rotating shaft. The liquid on this open, lip-side of the seal may be in contact with the intersection of the contact lip 169 and the rotating shaft 140, and a very small amount of the liquid may form a layer between the lip 169 and shaft 140. If that liquid is blood, some constituents of the blood such as proteins may be affected by the mechanical forces or heat in this area causing them to coagulate or stick to the shaft, which may reduce the longevity of the seal material or the duration of functionality of the seal or pose a safety hazard to the patient. Features described in relation to FIGS. 9B, 10, 11, 13A and 13B, such as grease, a distal protection disc, a distal protection disc having a contoured face, and/or impeller proximal vanes may mitigate this risk.

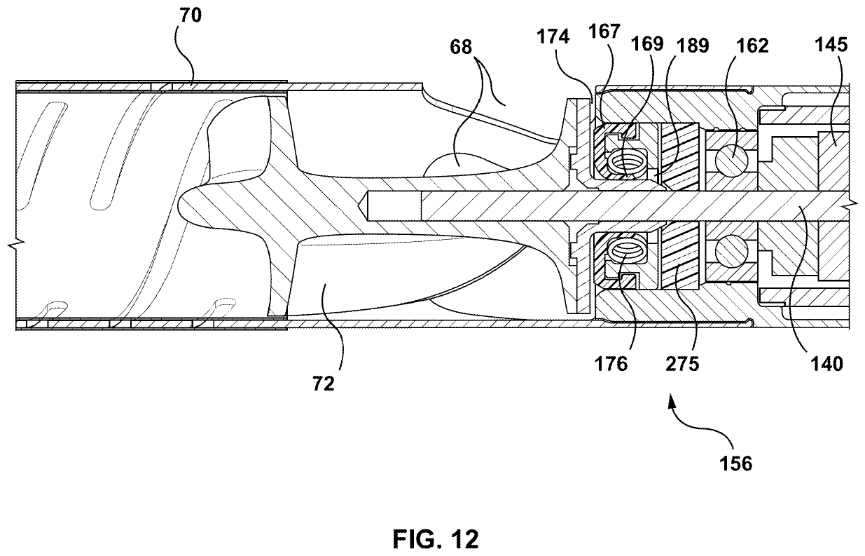

Conversely, orienting the seal in the opposite direction (e.g., such as the orientation shown in FIG. 12), with the lip and seal cavity facing away from the blood, and including a supply of lubricating grease in the seal cavity, the liquid that contacts the lip-shaft intersection may preferentially be the lubricant, which may slow down or eliminate the deposition of blood particles. It is with the discovery of this “reverse” or “backward” orientation of the seal that some of the embodiments described herein are based on.

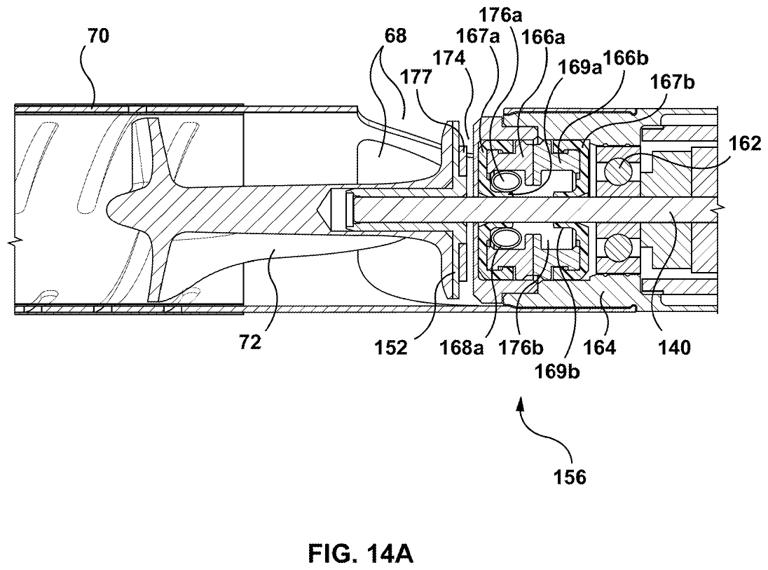

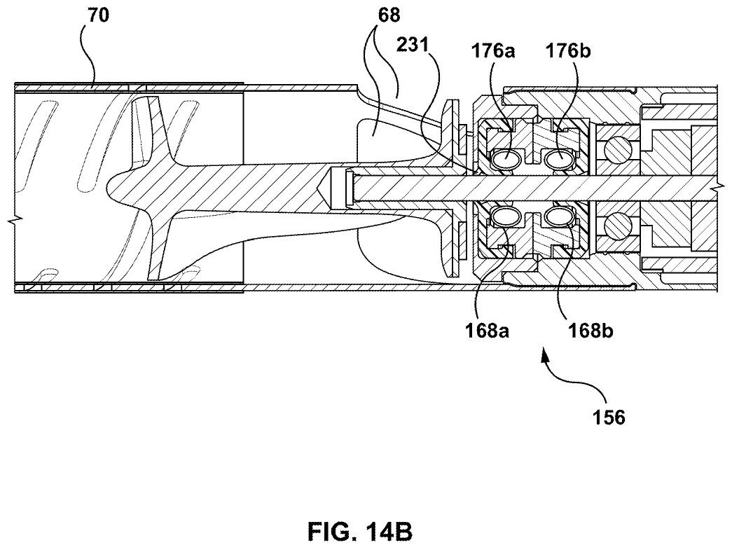

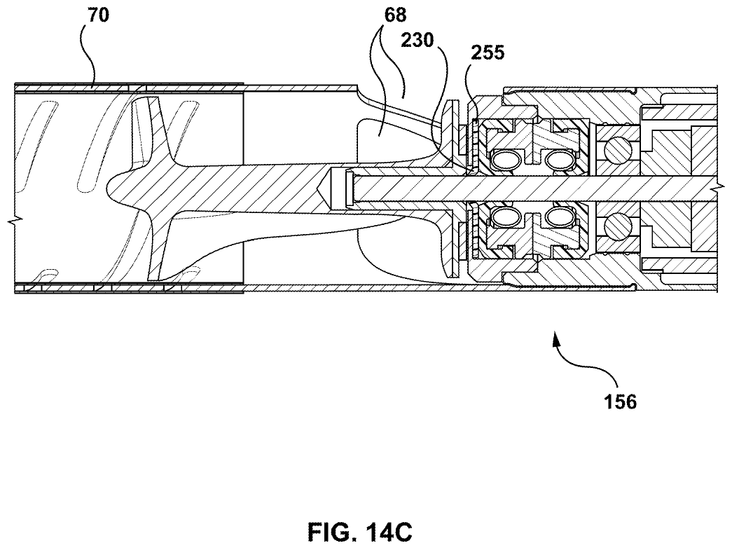

Further, the conventional approach to sealing is to “keep fluid out.” However, the seal configurations described herein may be designed based on the principle of “keeping lubricant in,” which in turn has the effect of being superior at keeping fluid (such as blood) out. For instance, including two seals facing one another, for example as shown in FIG. 14A, 14B, 14C, 15, 16A, or 19, may provide further advantages. Each seal cavity may function as a depository for a lubricant, such as grease. Two seal cavities facing one another may create a larger depository for grease and the seals may function to retain the grease in the depository and prevent or slow it from escaping, while the lubrication in the grease, which is designed to withstand mechanical or thermal stress, is the liquid that contacts the lip-shaft intersection, instead of blood. Thus, the sealing arrangement results in keeping blood out, based on the principle of keeping grease in.

### D. Embodiment with a Single Seal and Distal Disc

FIG. 10 is a cross-sectional view of another embodiment of an impeller region of an MCS device having an alternative seal configuration. This embodiment is similar to that of FIG. 9B with the exception that a distal protection disc 255 (also referred to as a distal disc 255) is disposed distal to the annular seal. The distal protection disc provides at least a partial barrier between the patient's blood and the annular seal assembly, which comprises a seal holder 166, an annular seal 167 with a seal lip 169, and a garter spring 168. The distal protection disc functions to reduce contact between blood and the seal by closing a large majority of an opening in the motor housing 164 where the seal is first inserted. Thus, the distal protection disc covers a large portion of the seal that is otherwise exposed to blood. The distal protection disc also creates a larger distance for blood to travel before it meets the seal and acts as a thermal insulator between blood and heat producing regions of the device, such as the motor or the seal, to reduce risk of blood damage or clotting. The distal protection disc 255 may be shaped like a circular disc with a central hole 171 through which the drive shaft may pass, and a thickness 172, for example a uniform thickness. It may fit tightly (e.g., hermetically scaled) against the motor housing 164 and therefor have a diameter equal to or slightly larger than the inner diameter of the motor housing to form a tight fit (e.g., friction fit). Optionally, the distal protection disc may have a form-fitting feature 173 such as a protruding ring around its outer circumference or a groove that mates with a form-fitting feature of the motor housing 164 for additional securement and scaling. Optionally, the distal protection disc may be adhered to the motor housing with adhesive or welding. The central hole 171 is sized to have a very small gap (e.g., a gap less than or equal to 0.05 mm, less than or equal to 0.01 mm) between the distal protection disc 255 and motor drive shaft 140, which may include an impeller proximal extension 154, passing though the central hole. In some embodiments, the distal protection disc does not contact the drive shaft 140 passing through the central hole, which ensures no additional friction is created or no additional torque loss is created. For example, the central hole 171 may have a diameter that is equal to the diameter of the drive shaft (140) plus twice the small gap (e.g., if the drive shaft has a diameter of 0.6 mm the central hole may have a diameter in a range of 0.62 to 0.70 mm). Optionally, a portion of the central hole may have an inner diameter that is less than the outer diameter of the drive shaft to make contact and function as a barrier to fluid. The thickness 172 may be in a range of 0.1 mm to 1.5 mm (e.g., 0.3 to 1.2 mm, about 1 mm). The distal protection disc may be made from a polymer such as PEEK, PTFE, or an elastic polyurethane, which may beneficially function as a thermal insulator, allow slight deformation when fitting into the motor housing, or may minimize friction in the situation where the drive shaft (140) temporarily or inadvertently contacts the disc. Furthermore, a slippery surface of the material may enhance flow of blood in an axial gap 174, the space between the impeller 72 and the stationary components facing the impeller such as the motor housing 164 or distal protection disc in this case. Alternatively, a distal protection disc may be made from a metal such as titanium or steel. Another function of the distal protection disc 255 is to contain a lubricating grease 175 in a seal cavity 176.

A seal cavity 176 is a volume of space within the seal where grease may be stored. For example, a seal cavity 176 as shown in FIG. 10, may be a volume of space defined by a seal holder 166, an annular seal 167, and a distal protection disc 173. The garter spring 168 may also be contained within the seal cavity 176. In the configuration shown in FIG. 10 the seal cavity 176 is facing distally, i.e., toward the impeller.

As shown in FIG. 10, the impeller 72 may optionally be connected to an impeller base plate 152, and the impeller base plate may optionally have proximal vanes 177. Alternatively, an impeller may be directly connected to a motor drive shaft 140, and the device may be with or without proximal vanes.

A method of manufacturing the device shown in FIG. 10 may include dispensing the grease into the seal cavity 176 that contains the garter spring 168 prior to assembling the seal components (e.g., seal holder 166, annular seal 167, spring 168) into the motor housing 164. To completely fill the seal cavity and encompass the spring in grease the seal components containing dispensed grease may be spun in a centrifuge or depressurized in a vacuum chamber to remove air bubbles. The seal components may then be pressed into the motor housing and additional grease may be applied in the seal cavity or distal to the annular seal 167 before covering with the distal protection disc 255.

### E. Embodiments with a Single Seal with Distal and Proximal Discs

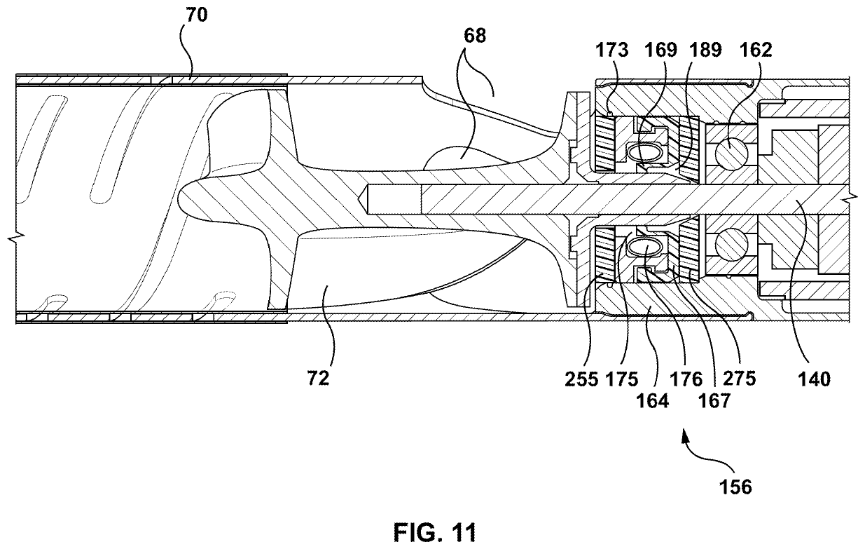

As shown in FIG. 11, an MCS device may have both a distal protection disc 255, for example as described in relation to FIG. 10, and a proximal disc 275 (also referred to as a proximal disc) disposed adjacent and proximal to the annular seal 167, and distal to the motor bearings 162 and motor. The proximal disc 275 may function to reduce contact between the motor bearings or motor and blood by sealing a majority of the pathway and creating larger distance for blood to travel before it meets the motor. For example, in the event that blood manages to pass the distal protection disc 255 and the annular seal 167, the proximal disc 275 may act as an additional measure to prevent blood from passing further into the motor housing. A combination of a distal protection disc 255 distal to one or more annular seals 167 and a proximal disc 275 proximal may restrict or reduce blood from passing from the external environment to the motor, may reduce thermal transfer from the motor or bearings 162 to the annular seal 167 or to the blood in the external environment, or to blood contacting surfaces.

Another benefit is that the proximal disc 275 together with the annular seal may define a proximal cavity 189 on a proximal side of the annular seal 167 in which a second storage of lubricant or grease may be located. Optionally, a first storage of lubricant or grease may be located in the seal cavity 176, which in this case is on the distal side of the annular seal 167. Having a first and second storage of lubricant or grease on each side of the annular seal may further reduce friction between the annular seal and drive shaft by providing a larger volume of grease or by providing grease on each side of the annular seal to ensure there is a continuous layer of grease between the annular seal lip 169 and moving parts interacting with the lip such as a drive shaft 140 or impeller proximal extension 154 thus increasing the duration of seal integrity. Optionally, the first lubricant contained in the seal cavity 176 and the second lubricant contained in the proximal cavity 189 may be different substances. For example, the first lubricant may be a higher consistency grease (e.g., NLGL grade 3 to 4), which may function to remain mostly contained in the seal cavity 176 and surround the garter spring 168 at least for a duration of use to prevent blood from entering the garter spring 168. The second lubricant may be a relatively low consistency grease, which may function to primarily lubricate the seal lip 169. A portion of the second lubricant may also contact the seal lip 169, the interacting moving surface, or the distal protection disc to provide a low friction interaction. Alternatively, the same grease may be used in the seal cavity 176 and the proximal cavity 189.

A method of manufacturing the device of FIG. 11 may include the grease dispensing steps described above in relation to FIG. 10, with an additional step of pressing the proximal disc 275 into the motor housing 164, and dispensing the first storage of grease or lubricant into the proximal cavity 189, prior to pressing the seal components into the motor housing.

### F. Embodiments with a Single Reversed Seal and Proximal Disc

Another implementation of an MCS device having a sealed rotary shaft is shown in a cross-sectional illustration in FIG. 12, wherein the rotary shaft lip seal 167 is oriented with its contact lip 169 and seal cavity 176, or its “open” side, facing proximally, i.e. toward the sealed motor 145 and away from the impeller 72. The opposite distal side, which may be flat as shown, faces the fluid side and impeller. A proximal disc 275 is positioned proximal to the lip seal 167 and a lubricating grease is deposited in a space defined by the seal cavity 175 and the proximal disc 275. Optionally, the MCS device may have an impeller with proximal vanes to increase blood flow in the axial gap 174, which may beneficially remove heat from the seal contact region to reduce a risk of blood coagulation. Optionally, the lip seal 167 may have a leading edge 231 (see FIG. 14B) to further prevent blood from passing the seal.

### G. Embodiments with a Distal Protection Disc and Contoured Flow Surface

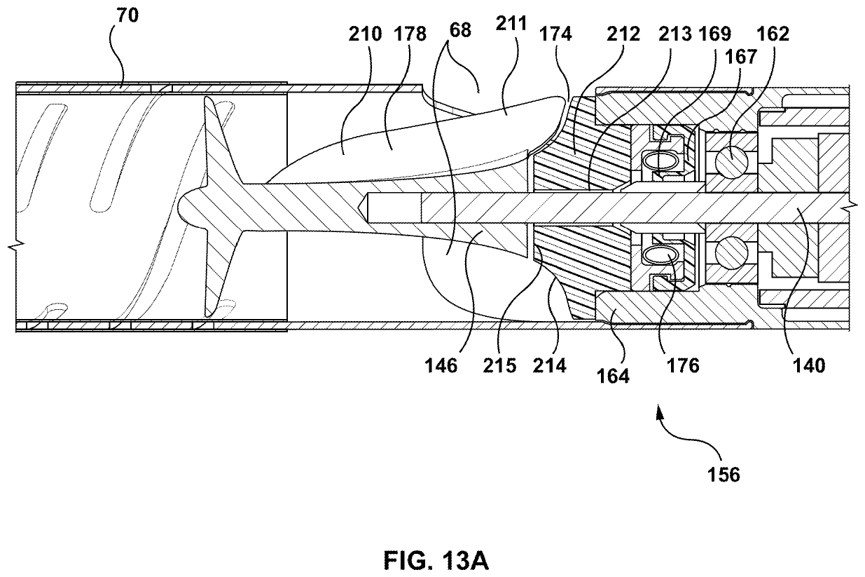

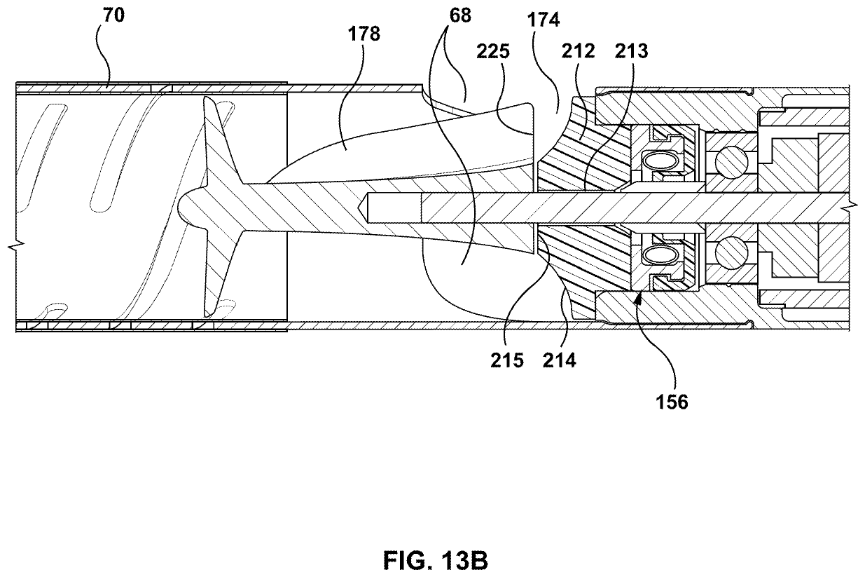

Another implementation of an MCS device having a sealed rotary shaft is shown in a cross-sectional illustration in FIG. 13A. As shown, the cross-sectional view of the MCS device has a distal facing lip seal 167 and a distal protection disc 212 having a distally facing conical surface 214 with a concave contour and an impeller 210 with blades having proximal regions 211 that match the contoured face. The distal protection disc 212 may be made from an elastomeric material such as PTFE, PEEK or a compound and have a center bore 213 with an inner diameter that is slightly larger than the rotary shaft position within the center bore so contact is minimized or avoided. For example, a radial gap between the rotary shaft 140 and the distal protection disc 212 may be in a range of 40 μm to 75 μm (e.g., about 0.05 mm). Optionally, the distal protection disc may make a contact with the rotary shaft at least partially. Optionally, at least a portion of the central opening 213 has a diameter that is less than the outer diameter of the drive shaft 140, optionally by a difference in a range of 0.01 to 0.05 mm. The distal protection disc 212 may protrude from the motor housing 164 (e.g., protrude by a distance in a range of 1 to 2 mm), which may increase the distance between the seal 167 and flowing blood, which in turn may prevent blood from entering the seal for a longer duration or thermally insulate the blood from heat generated in the motor. The protrusion of the distal protection disc may have a contoured surface, for example a tapered or conical portion 214 optionally with a concave surface and a flat surface portion 215. The flat surface portion 215 may have a diameter equal to or similar to (e.g., within 0.01 mm) the diameter of the flat portion of the base of the impeller 210. The shape of the tapered portion 214 may transition smoothly from the shape of the impeller hub 146, which may facilitate directing blood flow from the inlet tube 70 and out the outlet windows 68. A narrow axial gap 174 is between the impeller 210 with blades having proximal regions 211 that match the contoured face.

Alternatively, as shown in FIG. 13B the MCS device has a distal facing lip seal and a distal protection disc having a contoured face may have an impeller 72 with a non-matching proximal shape, such as a flat proximal edge 225.

An alternative implementation may have a distal protection disc having a contoured face and an impeller with a matching contour as shown in FIG. 13A or an impeller with a non-matching contour as shown in FIG. 13B, but with a proximally facing lip seal and optional proximal disc.

### H. Embodiments with Two Radial Shaft Seals

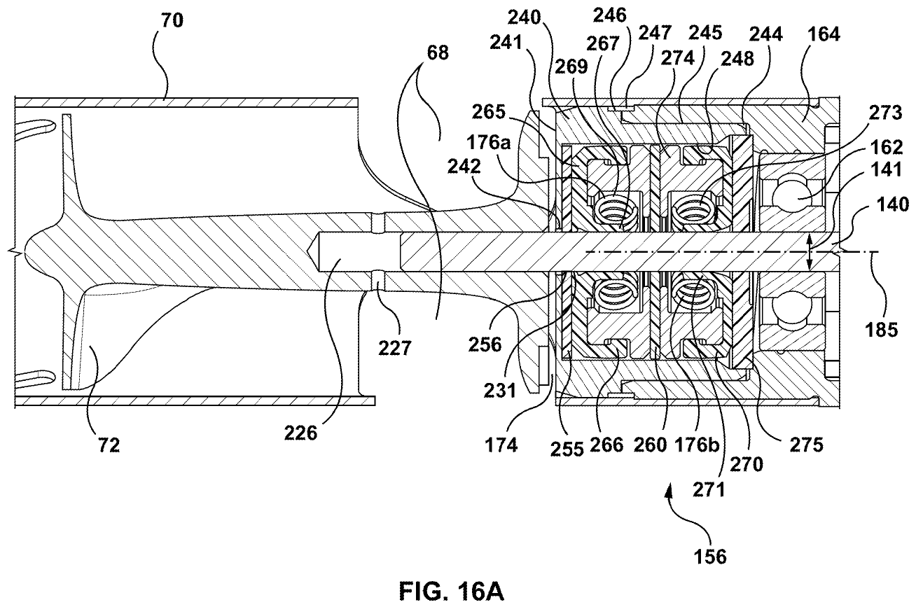

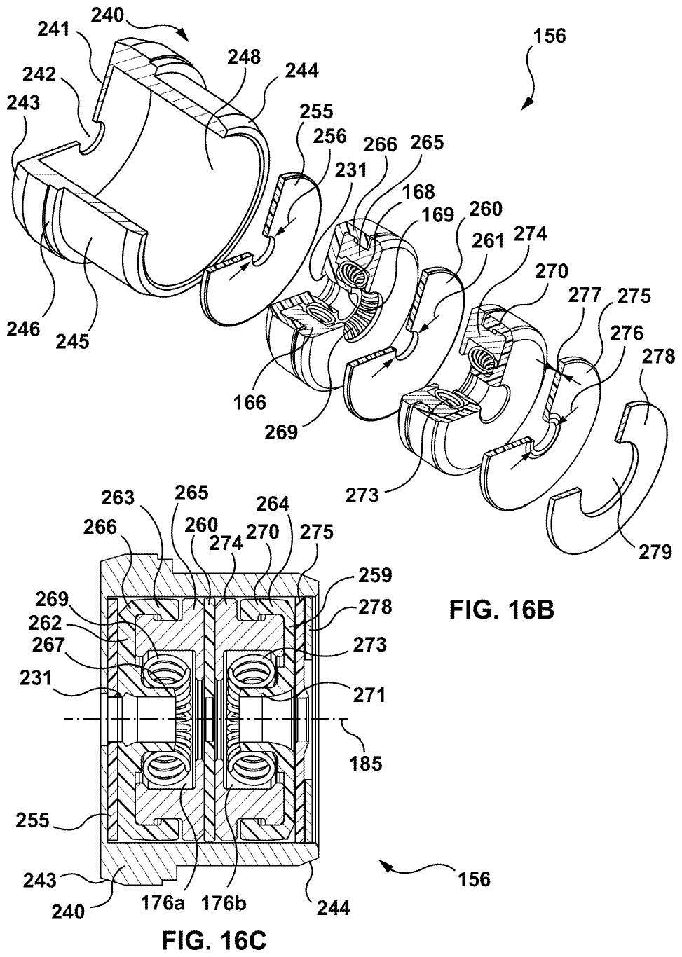

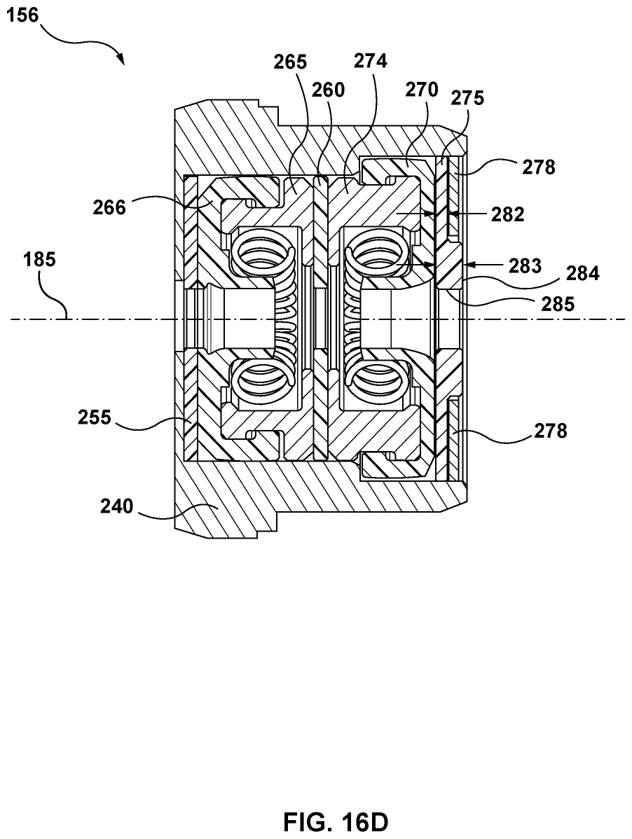

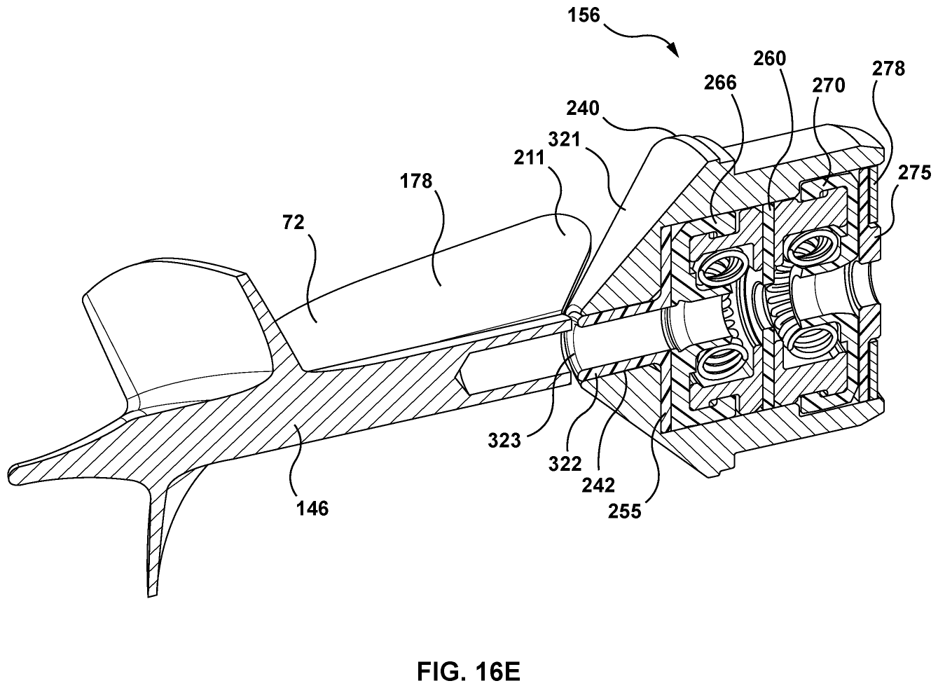

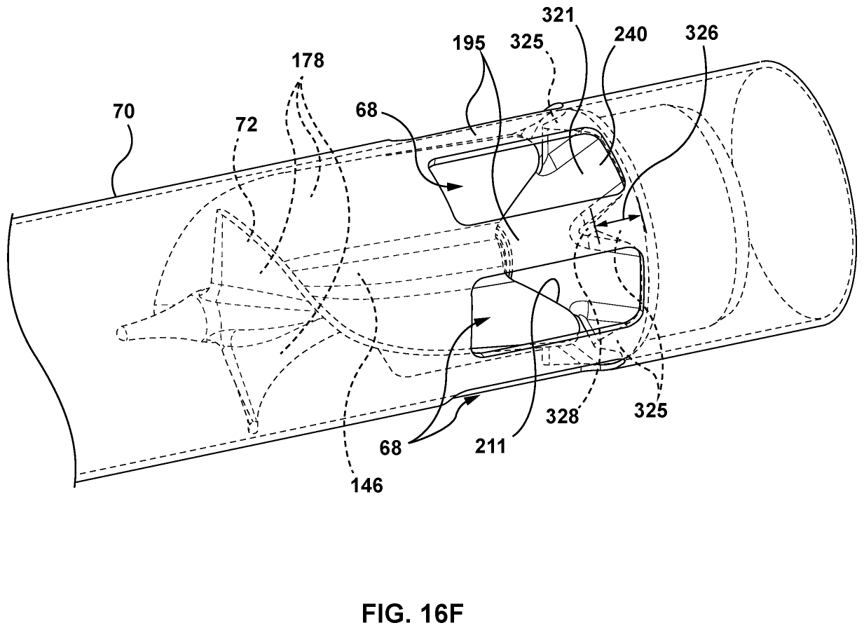

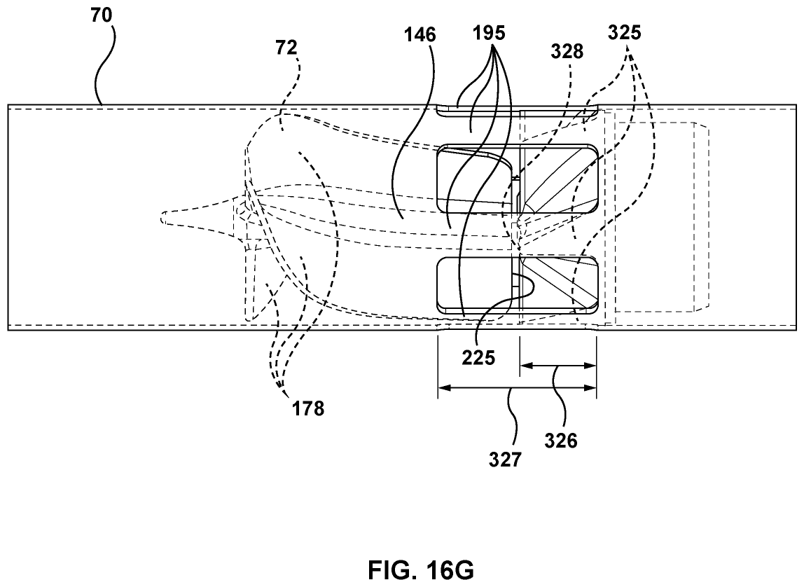

Other embodiments of MCS devices having a sealed rotary shaft are shown in FIGS. 16A-16G. FIG. 16A is a partial cross-sectional view of an MCS device having two lip seals (aka radial shaft seals) facing one another, a distal disc, a middle disc, and a proximal disc contained in a seal housing. FIG. 16B is an isometric, exploded, partially cut-away view thereof, and FIG. 16C is a cross-sectional view of the seal components shown isolated as a subassembly. FIG. 16D shows an alternative embodiment of a seal assembly having a proximal disc with an extended radial contact surface and an axial contact surface, FIG. 16E shows an embodiment of an MCS device having a sealed rotary shaft and tapered container, and FIGS. 16F and 16G show an embodiment of an MCS device having a sealed rotary shaft and tapered container with output struts, as further described herein.

As shown in FIGS. 16A-16C, the device includes a distal annular radial or rotary shaft seal 266 having a radially inward contact lip 267 forming a seal cavity 176a. The contact lip 267 and seal cavity 176a of the distal seal 266 faces proximally. The distal seal 266 thus has an “open side” facing proximally toward the motor, and a “flat side” facing distally toward the impeller and blood. The distal seal 266 is thus oriented “backwards” from conventional orientations. In some embodiments, the “open side” may be a side of the seal 266 formed in part by upper and/or lower flanges or lips of the seal 266. A cavity may be formed by the open side of the seal 266. The cavity may be formed between an end wall of the seal 266 and the one or more flanges or lips of the seal 266. The cavity may have a spring and/or grease located therein. Further details of the end wall, lips, etc. are described herein.

The device further includes a proximal annular radial or rotary shaft seal 270, having a radially inward contact lip 271 forming a seal cavity 176b. The contact lip 271 and a seal cavity 176b of the proximal annular seal 270 faces distally. The proximal seal 270 thus has an “open side” (as described above) facing distally toward the motor, and a “flat side” facing proximally toward the impeller and blood. Therefore, the seal assembly includes the proximal annular seal 270 and the distal annular seal 266 having opposite orientations, with their contact lips 267, 271 and seal cavities 176a, 176b facing one another.

The lips 267, 271 contact the shaft 140. The lips 267, 271 may extend along the shaft 140. All or a part of the radially inward surface or surfaces of the lips 267, 271 may contact the shaft 140. The lips 267, 271 may be flat, and/or have non-flat features, as described in further detail herein, for example with respect to FIG. 16C.

The seals 266, 270 may include radially outer lips 263, 264. The lips 263, 264 may contact a radially inward surface of the housing or other component of the seal compartment. The lips 263, 264 may extend along the housing or other component. The lips 263, 264 may seal off the space between the seal 266, 270 and the housing or other component. The radially outer surfaces of the lips 263, 264 may be flat, non-flat, or combinations thereof.

The lips 263, 264 may extend from respective end walls 262, 259. The lip 263 extends distally from the end wall 262. The lip 264 extends proximally from the end wall 259. The end walls 262, 259 may refer to the “flat” sides described herein. The radially inner lips 267, 271 may extend from the end walls 262, 259, as described. The outer lips 263, 264 may extend perpendicular to the end walls 262, 259, either under no external forces and/or when installed in the seal compartment. The outer lips 263, 264 may have the same or similar features as the inner lips 267, 271, such as the leading edge, groove or recess, etc.

In some embodiments, a middle elastomeric disc 260 may be positioned between the proximal annular seal 270 and the distal annular seal 266. A distal elastomeric disc 255 may be positioned distal to the distal annular seal 266. A proximal elastomeric disc 275 may be positioned proximal to the proximal annular seal 270.

Optionally, a seal housing made of a distal seal container 240 and an optional seal container cap 278 (see FIGS. 16B, 16C, and 16D), may contain the seal components in a subassembly. The subassembly may be inserted over the drive shaft 140 and into a motor housing 164. Alternatively, the seal components may be assembled in the motor housing by inserting the components separately and sequentially over the drive shaft 140 into a cavity in the motor housing. The seal components may then be covered with a proximal (proximal) seal cap 278 that may be attached (e.g., welded, friction fit, form fit, glued) to the motor housing.

Both the distal elastomeric disc 255 and the middle elastomeric disc 260 may be made from an elastomeric, biocompatible material such as PTFE, an elastic polyurethane, or a compound material such as PTFE and Polyimide. As shown in FIG. 16B, one or more of the discs 255, 260 may have an inner diameter (ID) 256, 261 that is less than the outer diameter (OD) of the drive shaft 140, which optionally may include an impeller proximal extension 154 such as that shown in FIG. 10, that the inner diameter contacts. For example, the ID 256, 261 may be in a range of 80% to 95% (e.g., about 87%) that of the OD 141. In one implementation, the ID 256, 261 is 0.52 mm+/−0.02 mm and the OD 141 is 0.60 mm+/−0.01 mm. This dimensional difference creates high interference between the elastomeric discs 255, 260 and drive shaft to maintain a seal. For example, an ideal interference may be in a range of 0.070 mm to 0.080 mm The elastomeric discs 255, 260 may both have a thickness in a range of 80 μm to 140 μm (e.g., about 100 μm).

The properties of the elastomeric discs 255, 260 such as high interference, material durometer (e.g., in a range of 70 to 85 Shore), and thickness, may allow for the disc to deform when inserted over the drive shaft. For example, the disc may compress outward such that the disc ID may stretch, or the plane of the disc may curve particularly in a region close to the ID. The deformation of the disc may provide a contact pressure with the drive shaft 140 even as the disc material wears over time. Furthermore, the high interference provides an amount of material that may be worn down before contact pressure is reduced to zero, which may prolong the functional duration of the disc 255, 260 to act as a blood barrier. Furthermore, the high interference may compensate for small tolerances of eccentricity of the drive shaft within the disc.

The properties of the discs 255, 260 may allow them to act as a fluid barrier, at least for a portion of the intended duration that the MCS device is in use, while minimizing friction or decrease in torque transmission. Additionally, the distal elastomeric disc 255 may function as a first barrier to blood at least for a portion of duration of use. The middle elastomeric disc 260, may function as an additional barrier to blood if it manages to pass the more distal barriers. Also, the disc 260 may act as a divider between the distal annular seal cavity 176a and proximal annular seal cavity 176b help to keep grease that is contained in these cavities next to each annular seal, which in turn prolongs the functional duration of the annular seals. Optionally, the grease or lubricant dispensed in the distal seal cavity 176a may be the same or different than that dispensed in the proximal seal cavity 176b. In some embodiments, the proximal disc 276 may have the same or similar features as the distal and middle discs 255, 260.

Other than their relative position and orientation, the distal seal 266 and proximal seal 270 may have similar properties to one another or to other seals 156 disclosed in relation to other implementations. For example, both the distal and proximal seals may have a seal holder 265, 274, an annular seal with a contact lip 267, 271, a seal cavity 176a, 176b, partially defined by the seal holder and annular seal, and/or a garter spring 269, 273 held in the respective seal cavity 176a, 176b. The seals 266, 270 may have the same inner diameter and lip dimensions. Optionally the seals 266, 270 may have different outer diameters primarily so they are easily distinguishable from one another during manufacturing.

Alternative to a garter spring 269, 273 the seals may contain a different component that applies radially inward force such as an O-ring or not have a separate component that applies the force, wherein properties of an elastomeric annular seal with a contact lip self-applies a radially inward contact force.

The distal and proximal annular seals 266, 270, may be made from a biocompatible elastomeric material such as PTFE, an elastic polyurethane, or a compound material such as PTFE and Polyimide, which optionally may have one or more additives to enhance durability. Grease may be contained in one or both seal cavities 176a, 176b, and optionally a third grease reservoir held between the proximal seal and proximal disc 275, and may be the same grease or different greases. In one implementation a first grease is deposited in the distal seal cavity, which may have a higher viscosity and grease consistency (e.g., NLGL Class 4 or higher) than a third grease (e.g., NLGL Class 2) deposited in the proximal seal cavity or a second grease held in the third grease reservoir held between the proximal seal and proximal disc. In another implementation grease is deposited in the distal seal cavity (e.g., NLGL Class 4 or higher) and an oil is deposited in the proximal seal cavity.

Optionally, the distal seal 266 may have a leading edge 231 on its distal face, which in addition to the contacting lip 267 is a surface of the distal seal that contacts rotating parts such as the drive shaft 140. The leading edge 231 is a portion of the distal annular seal 266 with an inner diameter that is less than the inner diameter of a portion of the contacting lip 267 located proximally of the leading edge 231. The leading edge 231 may be a portion of the distal annular seal 266 with an inner diameter that is less than the outer diameter of the motor drive shaft 140 that the inner diameter mates with. For example, the ID of the leading edge may be in a range of 75% to 95% (e.g., 80% to 90%, about 87%) that of the OD 141. In one implementation the ID is 0.52 mm and the OD 141 is 0.60 mm. By making a flush connection to the rotating shaft 140 on the distal face of the seal, the leading edge may function to reduce the occurrence of blood getting actively drawn underneath the contacting lip 267, which may contribute to increasing the longevity of the seal. The distal annular seal 266 may be made as shown with a groove between the leading edge 231 and contact lip 267. The leading edge 231 may be formed in part by an adjacent groove or recess formed in the inner surface of the lip 267. Alternatively, the leading edge 231 may have a smooth transition to the contact lip 267.

The orientation of the proximal seal 270, wherein the contact lip 271 and seal cavity 176b are directed distally, may facilitate the overall sealing function in a number of ways: for example, lubricating grease is held in the cavities 176b and 176a between the distal seal 266 and proximal seal 270 which coats the contact surface between the contact lips 267, 271 and the drive shaft 140 to reduce wear, minimize reduction of torque transmission or heat formation, and resist ingress of blood; a higher pressure on the distal side of the seal 270 relative to the proximal side (e.g., due to compressed grease held in the seal cavity 176b or in the event that blood manages to pass through the more distal blood barriers) may support the contact pressure of the contact lip 271. The axial length of a portion of the contact lip 271 that contacts the shaft may be in a range of 0.3 to 0.8 mm (e.g., about 0.5 mm).

Optionally, the device may have the proximal disc 275 positioned proximal to the proximal seal 270 as shown in FIG. 16A. The proximal disc may function as another barrier to prevent blood from entering drive shaft bearings 162 or the motor compartment. Furthermore, the proximal disc may help to account for small tolerances in eccentricity of the drive shaft. The proximal disc 275 may be made from a biocompatible elastomeric material such as PTFE or an elastic polyurethane or a compound and have a generally disc shape with a center hole having an inner diameter 276 through which the drive shaft 140 passes and makes contact. The ID 276 may be in a range of 80% to 97% (e.g., about 93%) that of the OD 141. In one implementation the ID is 0.56 mm and the OD 141 is 0.6 mm, which may be greater than the ID of the distal disc 255 or middle disc 260 to have less impact on torque transmission losses. Optionally, the proximal disc 275 may have a greater thickness than the distal or middle discs 255, 260 as shown in FIG. 16A, which together with the elastomeric properties of the disc may provide an axial compression of the sealing components when the proximal disc is compressed between a distal seal container 240 and an edge on the motor housing 164. For example, the thickness of the proximal, middle and distal discs may be in a range of 0.10 mm to 0.15 mm. The proximal disc 275 may be axially compressed due to dimensions of the stack up of seal components in the axial direction and the space within the housing that compresses the stack. In some embodiments, the proximal disc 275 may be non-flat, e.g. spherical, such as a Belleville washer shape, to provide compression.

FIGS. 16B and 16C show the device of FIG. 16A but having a relatively thinner the proximal disc 275, as well as the addition of a seal container cap 278. In this implementation all of the sealing components are contained within a seal container, for example as a subassembly. The seal container may include a distal seal container 240 and the seal container cap 278, which may be both made from a metal such as stainless steel or titanium and connected securely for example, with a friction fit, form fit, threading, or weld.

FIG. 16D shows a seal 156 with the same features and functions as the seal in FIG. 16B except as otherwise described. For example, the seal 156 of FIG. 16D includes the proximal disc 275 having a first thickness 282 in the axial direction and a second thickness 283 in the axial direction that is greater than the first thickness 282. The second thickness 283 may be located closer to the central axis 185 relative to the first thickness 282. The second thickness 283 may be at least as thick as a combination of the first thickness 282 and a thickness of the seal container cap 278. The second thickness 283 may be thicker than the combination of the first thickness 282 and the thickness of the seal container cap 278 so that a proximally facing, protruding surface 284 protrudes from the cap 278 in a proximal direction (i.e. to the right, as oriented in the figure). When assembled, the proximally facing, protruding surface 284 may slidably contact a distal facing surface of at least a portion of a drive shaft bearing 162 (see FIG. 16A) to provide another layer of sealing. The increased second thickness 283 allows for a greater radially inner surface 285 that provides slidable contact with the motor shaft 140, which may increase sealing performance.

Referring to FIG. 16B, the distal seal container 240 functions to contain the seal components with or without the seal container cap 278 and facilitate manufacturing. The distal seal container may have a flat, rigid distal surface 241 that provides a surface for mechanically pressing the seal components into the motor housing 164 while protecting the softer, more fragile seal components. The flat, rigid surface 241 also ensures the axial gap 174 between the surface 241 and impeller is consistent so blood in the axial gap is expelled, and the proximal face of the rotating impeller does not contact the seal components inadvertently. The surface 241 has a central hole 242, which has an inner diameter that is larger than the outer diameter of the drive shaft 140. For example, the hole 242 may have a diameter that is in a range of 0.080 mm to 0.150 mm (e.g., about 0.100 mm) greater than the outer diameter of rotating parts passing through the hole, which may function as a physical filter to prevent particles from escaping the container as a risk management measure. For example, the hole 242 may be in a range of 0.68 mm to 0.75 mm (e.g., about 0.70 mm) when the drive shaft has a diameter of 0.60 mm. In other words, a radial gap between the drive shaft and the container 240 may be in a range of 0.040 mm to 0.075 mm (e.g., about 0.050 mm). The distal seal container has cylindrical side walls with an inner surface 248 that functions to constrain the seal components ensuring there is no lateral movement, which could compromise the integrity or longevity of the seals. A proximal chamfer 244 facilitates insertion into the motor housing during manufacturing. A distal chamfer 243 facilitates insertion of an inlet tube 70, or alternatively an impeller housing 82 over the distal seal container 240. Furthermore, the distal seal container 240 may have a recessed outer surface 245 for inserting into the motor housing 164. An embodiment of a heart pump 22 having a seal element 156 as shown in FIG. 16A may have a motor housing with a length no greater 25.5 mm. With additional length added to the motor housing by the seal subassembly and an optional wiring module connected to the proximal end of the motor housing, the length of the motor housing may be extended to no more than 33 mm.

In another embodiment, as shown in FIG. 16E, an MCS device may have a seal 156 in the form of a seal assembly, wherein the distal seal container 240 does not have a flat distal face 241 as shown in FIG. 16A but instead has a distally tapering, e.g. conical, face 321. The distally tapering conical face 321 may have a straight slope (as shown in FIG. 16E) or alternatively may have a curved slope, for example a concave contour like the surface 214 shown in FIG. 13A, or a convex conical contour, or combinations thereof. Due to the inside geometry of a seal container having a conical distal face 321 compared to a flat distal face, a portion of the seal components may extend into the interior tapered part of the distal seal container 240. This may allow the length of a rigid motor housing to be shorter. A shorter rigid motor housing beneficially traverses a curve such as the aortic arch more easily.

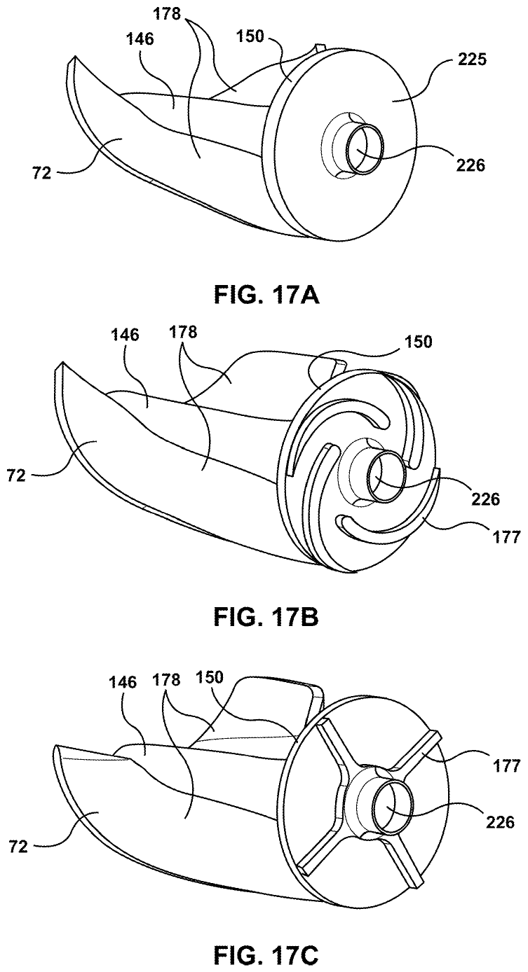

Further, the central hole 242 in the distal seal container 240 is longer in the conical-shaped container (FIG. 16E) compared to the flat-shaped container (FIG. 16D) and this extra length provides space for further sealing functions. The distal disc 255 may have a tubular extension 322 lining the inner surface of the central hole 242. The tubular extension 322 may be part of and be the same material as the distal disc 255, for example PTFE, and optionally may be adhered to the surface of the central hole 242. The inner surface of the tubular extension 322 may be configured to slidably engage with the motor shaft 140 to create another sealing function to further extend the duration of sealing performance. In some embodiments, the tubular extension 322 may have a surface texture or treatment on the inner surface intended to at least partially contact the motor shaft 140. A surface texture or treatment may be included on protruding circumferential ribs, indents, or a micropattern that may be hydrophilic or hydrophobic which may function to prevent passage of blood or hold lubrication. In some embodiments, the distal disc 255 and/or the tubular extension 322 may be made from a material that holds lubricant, such as felt. In some embodiments, a cavity may be made in the conical portion of the distal seal container 240 to hold lubrication. The tubular extension 322 may terminate so its distal axial face 232 is flush with the distal opening in the distal seal container 240, or it may extend beyond the container 240 and optionally make contact with the impeller hub 146 creating an axial face seal. The length of the tubular extension 322 that extends beyond the seal container 240 may be about 100 microns to provide an appropriate space between the impeller and distal seal container. In the configuration shown in FIG. 16E, the impeller 72 may not include an impeller base 150, such as shown in FIG. 17. Further, the impeller vanes or blades 178 may have a flat proximal edge 225 as shown in FIGS. 13B and 16G, or have a proximally extending proximal edge 211 following the distal contour 214, 321 of the distal seal container 240, as shown in FIG. 13A, 16E, or 16F. A “flat proximal edge” 225 may have an edge that is substantially orthogonal to the central axis. In some embodiments the flat proximal edges 225 or contoured proximal edges 211 of each impeller vane 178 may be connected to one another through an impeller hub 146 but not through an impeller base, such as the impeller base 150 shown in FIGS. 17A, 17B, and 17C, or impeller base plate 152 as shown in FIG. 14A. In other words, there may be an open space defined by the impeller blades 178 through which blood flow may be directed in an axial and proximal direction toward a distally tapered surface 321 where the blood flow is directed radially outward through the outlet windows 68.

FIG. 16F and FIG. 16G are perspective and side views respectively of different embodiments of a portion of an MCS device with the motor, motor housing, and motor shaft removed in order to illustrate the seal container 240, the impeller 72 and the inlet tube 70 more clearly. The inlet tube 70 is shown transparent to reveal the impeller 72 and the seal container 240. The MCS device of FIGS. 16F and 16G may include any of the seal assemblies described herein, such as the seal assembly of FIG. 16C, of FIG. 16D, etc.

FIGS. 16F and 16G show alternative embodiments of the distal seal container 240, further having outlet struts 195. As further shown, in some embodiments, the devices may include the outlet struts 195 with outlet strut supports 325. The outlet openings or windows 68 are openings in a cylindrical flow cannula, such as an inlet tube 70 or an impeller cage, that contains an impeller, which moves blood through the cannula from an inlet to the outlet windows. The outlet struts 195 are structures that hold the cannula to the motor housing, directly or indirectly. The outlet struts 195 may include one or two or three or four or more webs elongated axially and arranged radially about a longitudinal axis of the cannula. The outlet struts 195 may be made by laser cutting the outlet windows 68 in the inlet flow cannula 70 and the remaining material between the outlet windows may be the outlet struts 195, which may be substantially equal in geometry.

The outlet strut supports 325 may be connected to or machined as part of the distal seal container 240 and may each include a rigid structure spanning between the distal seal container 240 and an outlet strut 195, preferably on a position of the outlet strut between its proximal end and distal end, to provide increased rigidity and bending resistance to the outlet strut. The outlet strut supports 325 may have an axial length 326 that is a portion (e.g., up to 100%, up to 50%, up to 30%, about 30%) of the outlet strut length 327. The outlet strut supports 325 may have an axial length 326 that is a portion (e.g., up to 100%, up to 50%, up to 30%, about 30%) of the axial length of the conical portion 321 of the distal seal container 240. For example, FIG. 16G shows the outlet strut supports 325 having an axial length 326 that is 100% the length of the conical portion 321, and FIG. 16F shows the outlet strut supports 325 having an axial length 326 that is about 40% the length of the conical portion 321. The outlet strut supports 325 may contact or be adhered to the outlet struts 195 and be made of a rigid material that provides increased strength or resistance to bending to the outlet struts 195.

The outlet strut supports 325 may further function to direct the flow of blood from inside the outlet struts 195 toward the outlet windows 68. For example, the outlet strut supports 325 may have a surface that is angled in a direction of blood flow (i.e., from distal to proximal) from a location on a radially inner surface of an outlet strut 195 to a location on a radially outer edge of an outlet strut or in an outlet window 68 space. The outlet strut supports 325 may have a leading edge 328, that is configured to face upstream into the blood flow, and that may be rounded. In FIG. 16F, the leading edges 328 are positioned in the center of the widths of the outlet struts 195 and the outlet strut supports 325 each have a surface that is angled from the leading edge 325 to each adjacent outlet window 68 on each side of the outlet strut support. Alternatively, as show in FIG. 16G, the outlet strut support 325 may have a leading edge 328 positioned asymmetrically on an outlet strut 195, for example near an edge of an outlet strut, for example near an edge of an outlet strut facing a radial component of blood flow.

A method of manufacturing a seal subassembly may include but not be limited to inserting the seal components into the distal seal container in the order and orientation described herein, dispensing grease in the seal cavities optionally sequentially or simultaneously, releasing air bubbles using a centrifuge or vacuum chamber, and closing the seal container with the seal container cap 278. The seal subassembly may be inserted over a drive shaft 140, optionally into a motor housing, and connected to the motor housing, for example by laser welding an intersection which may include a rabbet 246 of the distal seal container 240 and a rabbet 247 of the motor housing. The impeller may be connected to the drive shaft, for example with an arrangement described herein in relation to FIG. 9B, 18A or 18B. An impeller housing 82 or an inlet tube 70 having an integrated impeller housing may be connected to the motor housing and/or distal seal container 240. The device may be packaged in an airtight package with air evacuated to prevent drying of the grease dispensed in the seals.

Alternative implementations of the concept shown in FIG. 16A are shown in FIGS. 14A, 14B and 14C, which have two rotary shaft lip seals oriented with their contact lips facing one another. The embodiments of FIGS. 14A, 14B and 14C may have the same or similar features as described with respect to the seal of FIGS. 16A-16C, and vice versa.

As shown in FIG. 14A, the seal may be without a distal, middle, and proximal disc. Optionally, only one of the two seals may contain a garter spring, which may allow for more grease to be deposited in the seal cavity 176b in addition to the seal cavity 176a containing a garter spring. The two seals may be inserted into the motor housing 164 and a rigid metallic cap may be positioned over the seals and welded to the motor housing. Alternatively, the two seals may be contained in a sealed container 240 like the one shown in FIG. 16C and inserted as a component into the motor housing.

FIG. 14B shows a similar implementation however having a garter spring in both the distal seal and the proximal seal. Furthermore, the distal seal has a leading edge 231 configured to contact the rotary shaft distal to the contact lip.

FIG. 14C shows another similar implementation however the distal seal does not have a leading edge. Instead, the device has an elastomeric distal disc 255 sized to contact the rotary shaft with a contact pressure, which may function as an additional barrier to blood.

### I. Embodiments with Pressure Balancing Features

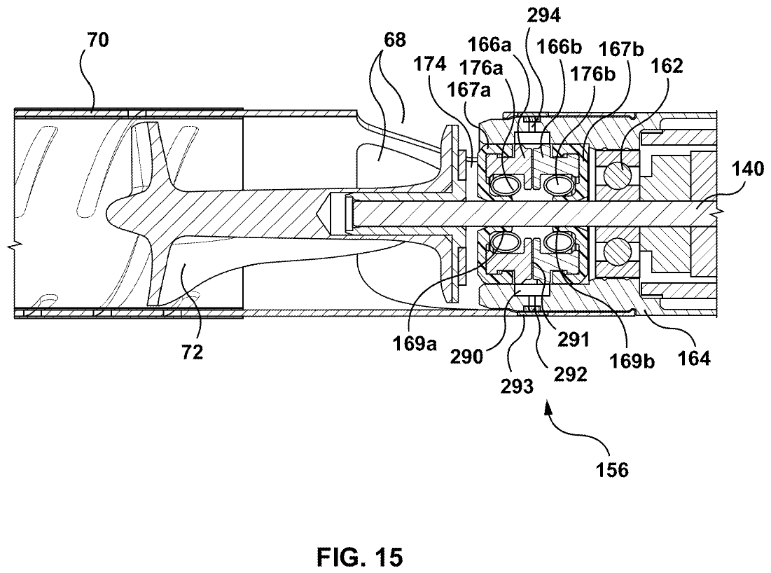

The embodiment of FIG. 15 includes pressure balancing features. Optionally, an MCS device with one or more rotary shaft seals, such as those shown in FIGS. 9B-16C. 18A, 18B or 19, may include the pressure balancing features.

The pressure balancing features may transfer or otherwise communicate pressure between the surrounding blood and the grease held in a seal cavity. Without being bound by theory, a pressure gradient between the pressure of surrounding blood applied to the external side of the seal components and the pressure of the grease applied to the internal side of the seal components may cause blood to enter the seal or lubricant to leave the seal. By balancing the pressures and decreasing or eliminating the pressure gradient, migration of blood or lubricant may be minimized. This may allow the device to prevent blood regress and extend the duration of functionality. A pressure balancing feature may be employed with any of the seal implementations disclosed within and may be used with one or two or more rotary shaft lip seals, one or more axial face lip seals, seal discs, seal containers, or with seals oriented distally or proximally, and in particular with at least one seal with its seal cavity oriented proximally.

For example, as shown in FIG. 15, pressure balancing features may be employed with two rotary shaft seals oriented with their seal cavities 176a, 176b toward one another. The pressure balancing feature includes at least one port or housing channel 294 providing pressure communication between the at least one seal cavity and an external environment that has the same or similar pressure characteristics as the external environment that is in contact with the seal, such as within the left ventricle. For example, a pressure balancing housing channel 294 may be positioned within 20 mm (e.g., within 15 mm, within 10 mm, within 5 mm) of the axial gap 174 so both the housing channel 294 and axial gap 174 are positioned in a left ventricle. A fluid tight, yet flexible, diaphragm 292 plugs each housing channel 294 to prevent passage of blood or lubricant but passively deforms to transfer pressure and decrease the pressure differential between the external blood and internal lubricant. If the ambient pressure in the heart increases above the pressure within the seal element, the diaphragm 292 will flex inward and cause an increased pressure inside the sealed cavity to provide higher resistance to blood ingress. Conversely, if the ambient pressure decreases below the pressure within the seal element, the diaphragm 292 will flex outward and cause a decreased pressure inside the sealed cavity to provide less outward force to the grease and lubricant stored therein.

As shown in FIG. 15, the seal cavity 176a, which optionally is joined to a second seal cavity 176b, may be filled with grease. The one or more seal cavities are in fluid communication with a seal channel 291 that is in fluid communication with a housing channel 294 that is in fluid communication with a diaphragm 292. Alternatively, a disc partitioner may be positioned between two seal cavities and one, preferably the distal seal cavity, may be in fluid communication with the channel (not shown). The seal channel 291 may be radial bores or passages in a seal holder or between two seal holders 166a, 166b. The housing channel 294 may be at least one (e.g., one, two, three, four, five, six) bores through one or more housings that contain the seals. For example, the housing channel 294 may be through the motor housing 164 as shown, or the seal housing if the seals are held in a container as shown in FIG. 16A. Furthermore, in implementations wherein an MCS device has an impeller housing or inlet tube 70 positioned over the motor housing 164, the housing channel may extend via a hole 293 through said impeller housing or inlet tube and the diaphragm 292 may optionally fill the hole 293. Alternatively, the hole 293 may be absent a diaphragm and have a smaller diameter than hole 292 that contains a diaphragm in order to anchor the diaphragm in place. The diaphragm may be silicone. Optionally, a lubricant reservoir 290 may be in fluid communication with both the seal channel(s) and the housing channel(s). The lubricant reservoir 290 may be made by machining an annular groove in the motor housing and may function to hold extra lubricant or disperse pressure around the seals evenly. Optionally, the lubricant reservoir may be filled with a different fluid than the grease, such as sterile water with glucose. Optionally, the lubricant has a viscosity in a range of 0.30 to 1.30 mPas.

A method of manufacture may include dispensing lubricant through at least one of the housing channels 294 before applying the diaphragms. A second housing channel that is in fluid communication with the seal cavities and the first housing channel may function as a vent to allow air to escape as lubricant is injected and may improve the ability to balance pressure.

Optionally, an axially compressible washer, such as a wave washer (not shown), may be positioned proximal to the proximal seal 167b and rest against a ridge or surface such as a seal container cap (not shown). The compressible washer may apply an axial force to the proximal seal and increase pressure in the seal cavities filled with grease or cause the diaphragm(s) to slightly bulge outward when the device is in atmospheric pressure or fluctuate about a relatively neutral position when exposed to blood pressure.

### J. Embodiments with Rotary Shaft Seal and Axial Face Seal with a Barrier Fluid

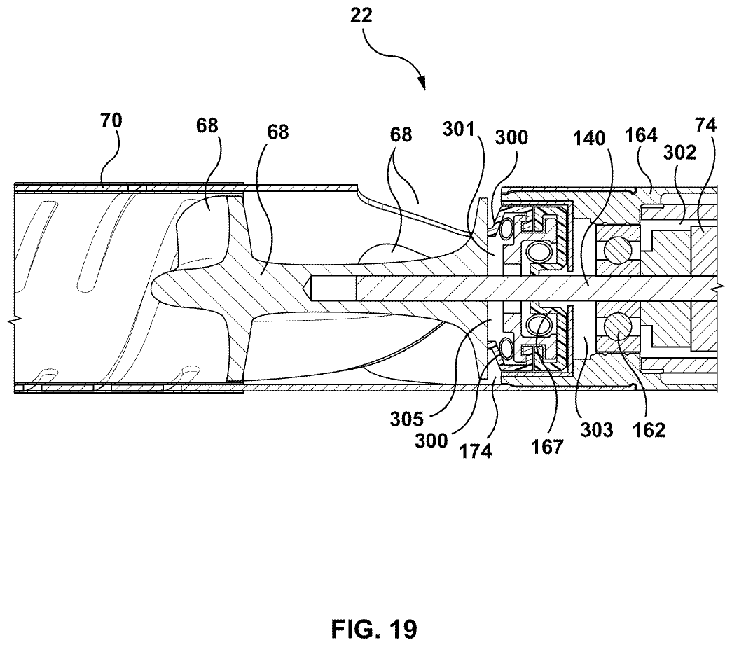

FIG. 19 shows a schematic representation of a heart pump 22 according to a design example. The heart pump 22 comprises a housing 164, an impeller 72, a motor 115, a sealing element 300 and a barrier fluid 301. The heart pump 22 represents a blood pump, typically an axial flow pump, which is driven by the motor 115 in the form of an integrated electric motor, and which generates a required blood flow by means of the impeller 72 when the heart pump 22 is implemented in the body of a patient.

The housing 164 has an interior 302 and an opening 303 to the interior 302. The interior 302 is shaped to accommodate the motor 115. The motor 115 is located in the interior 302 and has a shaft 140. The motor 115 is shaped to drive the shaft 140. The shaft 140 passes through the opening 303 and is coupled to the impeller 72 to drive the impeller 72. The impeller 72 has at least one blade 178, here exemplarily two blades 178, which are suitable for pumping blood. The impeller 72 is arranged on the drive shaft 140 that extends from the motor housing. The sealing element 300 is located between the impeller 72 and the motor housing 164 and is designed to seal the axial gap 174 between the impeller 72 and the motor housing 164. The sealing element 300 may be attached to the impeller 72. Alternatively, the sealing element 300 is attached to the motor housing 164. The sealing element 300 is ring-shaped to seal around the gap 174 completely. The sealing element 300 may be an axial face seal. Optionally, the heart pump 22 has a further sealing element 167, whereby the further sealing element 167 is located at the opening 303 and is designed to seal the interior 302 of the motor housing 164 against a space 305 between the motor housing 164 and the impeller 72. In this case, the space 305 is sealed by the sealing element 300 against the environment of the heart pump 22 and by the further sealing element 167 against the interior 302. Optionally the further scaling element 167 may be in the form or have other features of other radial rotary shaft sealing elements disclosed elsewhere herein such as a lip seal, or multiple lip seals.

According to this example, the barrier fluid 301 in the space 305 is held in the space 305 by the sealing elements 300, 174. The barrier fluid 301 prevents a medium from the environment of the heart pump 22 from penetrating into an interior of the motor 115. If the additional sealing element 167 is omitted, the space 305 is fluidically connected to the interior 302 of the housing 164. In this case, the barrier fluid 301 can expand into the interior of the motor 115. The motor interior can thus be flooded with barrier fluid 301.

The shaft 140 is mounted opposite the housing 164. For this purpose, two bearings 162 are arranged in the interior 302 as an example to support the shaft 140. According to one design example, during operation of the heart pump 22 blood, which can also be described as fluid, is fed axially to the impeller 72, sucked in here, and expelled radially and diagonally, for example through openings 68. The impeller 72 is fixed with the shaft 140 of the motor 115, which provides the required drive power. According to a design example, the shaft 140 is supported by at least one radial and at least one axial bearing 162. Optionally, the bearings 162 can also be used in combination with a radial-axial bearing 162.

The sealing element 300 can be contact or non-contact, e.g., as labyrinth seal or gap seal or as a combination of both. Furthermore, at least one additional sealing element 167 is optionally provided to seal the shaft 140 against the housing 164. The space between the sealing element 300 and the motor housing 164 is filled with the ideally biocompatible barrier fluid 301, which prevents the medium to be pumped (blood) from penetrating into the interior of the motor over the entire required operating time and service life of the heart pump 22. Optionally, the additional sealing element 167 reduces leakage of the barrier fluid 301 in the motor interior. According to another possible design example the complete interior of the motor is filled with the barrier fluid 301. The barrier fluid 301 ideally consists of a biocompatible medium, e.g., glucose or endogenous fat. Furthermore, viscosity of the fluid should be preferred, which neither causes too much friction loss nor, due to its low viscosity, evaporates from the space 305 during operation. Furthermore, good compatibility with the motor components should be sought.

For example, the heart pump 22 is available as a temporary or short-term Ventricular Assist Device (VAD) or mechanical circulatory support (MCS) pump, which can be implanted very quickly. For this purpose, the Heart Pump 22 is designed as a simple system according to a design example. The advantage is that although the Heart Pump 22 requires an external energy supply, it does not require an irrigation medium, which serves to protect the motor 115 from blood penetration. The heart pump 22 comes without a such external forced flushing.

According to one design example, the heart pump 22 essentially consists of the impeller 72 and the sealing element 300, which is firmly connected to the impeller 72 and has a sealing function against the housing 164, or alternatively a corresponding sealing element which is firmly connected to the motor housing 164 and has a sealing function with respect to the impeller 72. Furthermore, the heart pump 22 has an optional sealing element 167 which seals the housing 164 against the rotating shaft 178. A special feature here is that the space 305 between the two sealing elements 300, 167 is filled with the barrier fluid, which prevents the pumped medium (blood) from penetrating into the interior of the motor over the operating period.

### K. Embodiments with Surface Treatment

Optionally, a surface treatment may be applied to one or more parts to help prevent coagulation of blood or blood particles from sticking to the surface, or to facilitate movement of blood, or reduce friction between a seal lip or disc and a rotary shaft. For example, a surface treatment may be applied to the proximal surface of the impeller, the rotary shaft, the distal surface of a seal container 241. A surface treatment may be a hydrophilic coating such as Polyvinylpyrrolidon (PVP) having a thickness in a range of 3 to 5 μm, or a hydrophobic coating such as Perfluoralkoxy (PFA) having a thickness in a range of 10 to 20 μm. A surface treatment may be a micropatterned surface with hydrophilic or hydrophobic properties. A surface treatment or material used to make a component such as a seal holder 166, may include nitrided titanium, ceramic, or ceramic impregnated with PTFE.

### L. Embodiments with Superabsorber

Optionally an MCS device may contain a superabsorber in a rotary shaft seal assembly, such as those disclosed herein. For example, a superabsorber material may be provided on a carrier material such as a thin piece of foil or cellulose and positioned in a seal assembly such as in or in contact with a seal cavity. The carrier (not shown) may be in the form of a disc with a center hole and positioned on one or more of a distal side of the middle disc 260, a proximal side of the middle disc 260, or the distal side of the proximal disc 275 of the implementation shown in FIG. 16A. Alternatively or additionally, a superabsorber may be an encapsulated superabsorber granulate that is placed within a seal cavity, optionally mixed into the grease. The rotary shaft lip seal or other features such as a leading edge of a seal or a disc may provide the primary barrier to blood ingress, however in case some blood enters the seal a superabsorber may absorb the blood and prevent it from passing further into the motor or coagulating between the lip and rotary shaft. A hydrophilic behavior of the superabsorber may avoid absorbing oil from the grease. If blood is absorbed, the absorber may increase in volume and add pressure on the seal lip to further prevent blood from entering. A superabsorber may include a small amount (e.g., <50 microliters) of sodium polyacrylate.

### M. Embodiments with Impeller Proximal Vanes

An MCS device may have features of implementations disclosed herein and may optionally further include an impeller 72 having a central impeller hub 146, axial flow blades (e.g., two blades) 178 radially extending from the hub 146, an impeller base 150 at a proximal end of the hub 146 and radial flow blades 177 arranged on a plane perpendicular to the axis of the hub, and a central bore 226 in and coaxial with the hub. Optionally, the impeller may have proximal vanes 177 on the proximal face of the impeller base 150.

Without being bound by theory, proximal vanes are structural protrusions, or alternatively indentations, extending radially on the proximal surface of the impeller base that enhances fluid flow in an axial gap 174 when the impeller rotates, which may improve convective heat transport, improve efficiency, and reduce damage of blood cells. Heat is generated in the motor and bearings and via friction between the seal and rotary shaft and can cause blood particles to coagulate. Removing heat from this area may reduce the risk of blood coagulation. A small axial gap is preferable over a large gap, which can generate losses in efficiency and vortex areas, which in turn can build pressure, decrease efficiency and damage blood. The proximal vanes increase flow while allowing a very small axial gap, for example an axial gap in a range of 0.08 mm to 0.3 mm. Furthermore, the proximal vanes may increase the radial component of the mixed axial and radial blood flow to move blood out of the outlet windows 68 or may decrease fluid pressure in the axial gap 174 which may improve the function of the seal.

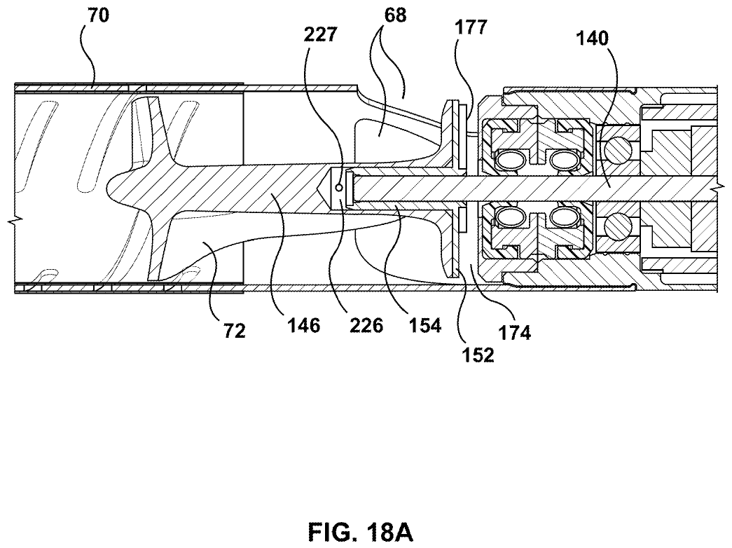

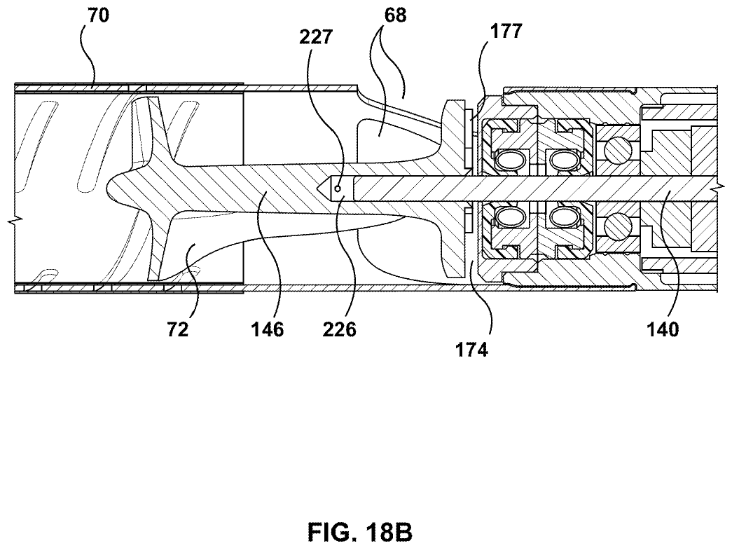

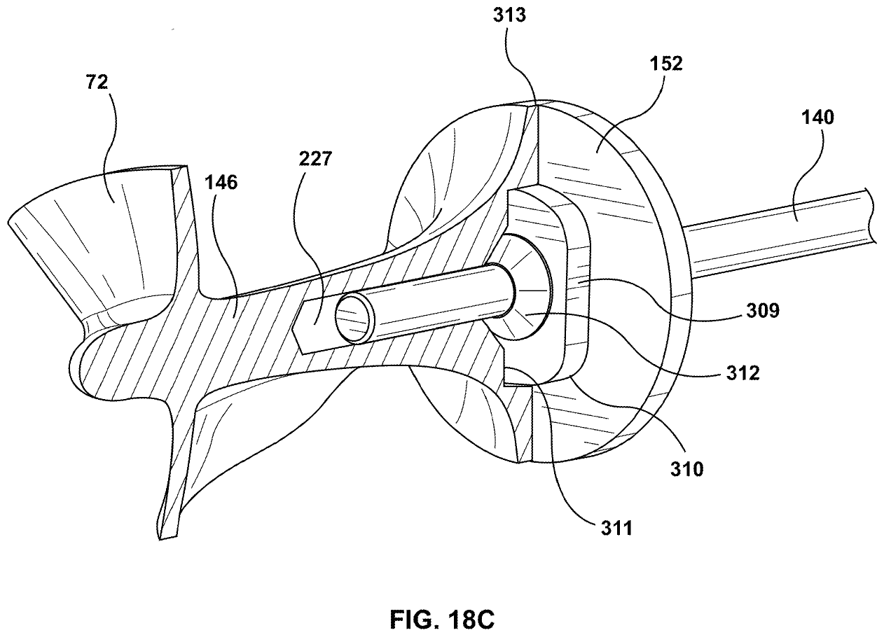

FIG. 17A shows an isometric schematic illustration of an impeller 72 of and MCS device that does not have proximal vanes but instead has a smooth proximal surface 225 on the impeller base 150. In contrast, FIGS. 17B and 17C show impellers with two versions of proximal vanes 177. Preferably, the impeller is balanced about its central axis so it rotates smoothly without vibration. To balance the impeller the proximal vanes 177 may be made to be radially symmetric, for example at least two proximal vanes on opposing sides of equal weight and shape may provide radial symmetry; three proximal vanes positioned 120 degrees about the central axis may provide radial symmetry; four proximal vanes positioned 90 degrees about the central axis may provide radial symmetry as shown in FIGS. 17B and 17C. As shown in FIG. 17B the proximal vanes 177 may be in a plane parallel to the impeller base 150 and be curved to encourage radially outward flow of blood. For example, the curvature of the proximal vanes may be convex in the direction of rotation. Alternatively, the proximal vanes may be straight and extend radially from the center bore 226 as shown in FIG. 17C. The proximal vanes may optionally have inner edges that are not connected to one another as shown in FIG. 17B or may be connected to one another as shown in FIG. 17C. Impeller proximal vanes 177 may be fabricated, for example machined or molded, directly on an impeller base 150 as shown in FIG. 18B, or alternatively be fabricated on a separate component such as an impeller base plate 152 that is connected to the impeller as shown in FIG. 18A.

### N. Embodiments of Connecting the Impeller to the Drive Shaft

The impeller 72 may be connected to the drive shaft 140 with a consistent axial gap 174 and in a rigid manner that reduces risk of contaminating or damaging the rotary shaft seal(s). In a first exemplary implementation as shown in FIG. 9B, an impeller 72 may be connected to a drive shaft 140 by first connecting an impeller base plate 152 to the drive shaft, wherein the base plate has a tubular proximal extension 154 extending proximally. This implementation is described above in detail. Optionally the base plate 152 may have impeller proximal vanes 177 on the proximal face of the base plate 152 facing into the axial gap 174.

In a second exemplary implementation as shown in FIG. 18A, an impeller base plate 152 having a tubular extension 154 may be first connected to the drive shaft 140. In contrast to the implementation of FIG. 9B the base plate 152 may be oriented with the tubular extension 154 aiming distally. Optionally, the tubular extension may have a non-circular cylindrical extension to rotationally lock the impeller to the tubular extension and base plate. The base plate may be connected to the drive shaft 140 by laser welding or adhesive. For example, a laser weld may be applied to the interface between the distal end of the tubular extension 154 and drive shaft 140 while a spacer is temporarily placed in the axial gap 174 to ensure a consistent gap and straight alignment. The impeller 72 may then be connected to the base plate 152 by inserting the tubular extension 154 into a central bore 266 in the impeller. The impeller 72 may be connected to the driveshaft 140, for example by dispensing glue in an impeller central bore 226 and sliding the impeller over the drive shaft wherein side bore(s) 227 allow air or excess glue to escape Optionally, the base plate may have impeller proximal vanes 177 on its proximal face aiming into the axial gap 174.

In a third exemplary implementation as shown in FIG. 18B, an impeller 72 may be connected directly to the drive shaft 140, for example without a base plate. The impeller 72 may be connected to the driveshaft 140, for example by dispensing glue in an impeller central bore 226 and sliding the impeller over the drive shaft wherein side bore(s) 227 allow air or excess glue to escape, while ensuring an axial gap 174 of a known distance, for example by implementing a manufacturing jig or inserting a spacer in the gap 174.

In a fourth exemplary implementation as shown in FIG. 18C, an impeller 72 and an impeller base plate 152 may be connected to a drive shaft 140 with a key 309. The impeller base plate 152 may optionally have a tubular extension oriented proximally and may optionally have impeller proximal vanes 117 oriented proximally into the axial gap 174. The impeller base plate 152 may have a recess 310 that the key 309 tightly fits into with a portion extending from the recess 310. The impeller 72 may also have a recess 311 in which the portion of the key 309 extending from the recess 310 may mate into. Recess 311 may have additional space to make room for a weld seam on the distal face of the key 309 and around the shaft 140. The key 309, recess 310 and recess 311 have mating shapes and are non-circular in the plane transverse to the axis of rotation so the key 309 cannot spin within the recesses but instead transfers rotational force from the shaft 140 to the impeller base plate 152 and impeller 72 through the key 309, at least in part. A method of assembly may include assembling a seal (e.g., a seal subassembly such as the one shown in FIG. 16A, 16B or 16C, or another implementation of a seal disclosed herein) over the drive shaft 140 extending from a motor; positioning the impeller base plate 152 having a recess 310 on the drive shaft while maintaining a desired axial gap 174, for example with a temporary spacer; positioning the key 309 over the drive shaft 140 and into the recess 310; laser welding the key 309 to the drive shaft 140 to create a weld seam 312; positioning the impeller 72 on the assembly so the drive shaft 140 is inserted into the impeller's central bore 226, the key 309 is inserted into the impeller's recess 311, and the base of the impeller is in contact with the impeller base plate 152. The impeller 72 may be laser welded to the base plate 152 to create a weld seam 313. Optionally, an adhesive may be applied in the impeller's central bore 226 before inserting the drive shaft in which case a side port 227 may allow excess adhesive or air to escape. Optionally, adhesive may be applied between the impeller 72 and impeller base plate 152, between the base plate 152 and shaft 140, or the key 309 and the impeller or base plate. Optionally, in this implementation the impeller 72 and the impeller base plate 152 may be made from different materials. In this case an adhesive may be used to bond them together with the key 309 held in the cavity between them and welded to the shaft 140.

### O. Modifications

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

### P. Terminology

It will be understood by those within the art that, in general, terms used herein are generally intended as “open” terms (e.g., the term “including” should be interpreted as “including but not limited to,” the term “having” should be interpreted as “having at least.” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### Q. Example Embodiments

The following is a non-exhaustive list of numbered example embodiments:

1. A seal for a heart pump, the seal comprising:
a distal radial shaft seal configured to surround a motor shaft of the heart pump, with a flat side of the distal radial shaft seal facing distally and an open side of the distal radial shaft seal facing proximally; and
a proximal radial shaft seal configured to surround the shaft and be located proximally of the distal radial shaft seal, such that the proximal radial shaft seal is located farther from an impeller of the pump than the distal radial shaft seal, with a flat side of the proximal radial shaft seal facing proximally and an open side of the proximal radial shaft seal facing distally.

2. The seal of Embodiment 1, wherein the distal radial shaft seal comprises a radially inner lip configured to contact the shaft and to extend from the flat side of the distal radial shaft seal in a proximal direction.

3. The seal of any of Embodiments 1 or 2, further comprising a distal spring located at least partially within the open side of the distal radial shaft seal and configured to compress a radially inner lip of the distal radial shaft seal radially inwardly onto the shaft.

4. The seal of any of Embodiments 1 to 3, further comprising a proximal spring located at least partially within the open side of the proximal radial shaft seal and configured to compress a radially inner lip of the proximal radial shaft seal radially inwardly onto the shaft.

5. The seal of any of Embodiments 1 to 4, further comprising one or more discs comprising a central opening with an inner diameter configured to be less than the outer diameter of the shaft.

6. The seal of Embodiment 5, wherein a radially inner edge of the central opening of each of the discs is configured to wear off in response to rotation of the shaft.

7. The seal of any of Embodiments 1 to 6, further comprising grease located between the distal radial shaft seal and the middle disc and between the middle disc and the proximal radial shaft seal.

8. The seal of any of Embodiments 1 to 7, wherein each of the distal and proximal radial shaft seals have radially outer lips configured to contact an inner side of a housing.

9. The seal of any of Embodiments 1 to 8, wherein the seal is configured to be assembled with the heart pump and delivered to the heart via a catheter.

10. The seal of any of Embodiments 1 to 9, further comprising a housing having a distal end wall and a cylindrical side wall extending proximally from the distal end wall, the distal end wall having a distal side configured to contact blood flow and having a central opening configured to receive therethrough the shaft, wherein the distal radial shaft seal is configured to be located proximally of the distal end wall at least partially within the housing.

11. A seal for a heart pump, the heart pump having a motor configured to rotate an impeller via a shaft about an axis, the seal comprising:
a distal radial shaft seal having a distal side configured to face distally toward the impeller and a radially inner lip configured to contact the shaft and to extend from the distal side in a proximal direction toward the motor.

12. The seal of Embodiment 11, further comprising a proximal radial shaft seal having a proximal side configured to face proximally toward the motor and a radially inner lip configured to contact the shaft and to extend from the proximal side in a distal direction toward the impeller.

13. The seal of any of Embodiments 11 to 12, further comprising one or more discs having an opening with an inner diameter that is less than an outer diameter of the shaft.

14. The seal of any of Embodiments 11 to 13, further comprising a seal housing configured to couple with a motor housing that is configured to support the motor, wherein the distal radial shaft seal is located at least partially within the seal housing.

15. The seal of Embodiment 14, wherein the distal radial shaft seal and seal housing are configured to be inserted as an integrated unit over the shaft.

16. A heart pump comprising:
an impeller;
a motor configured to rotate the impeller via a shaft about an axis; and
a seal comprising a distal radial shaft seal having a distal side configured to face distally toward the impeller and a radially inner lip configured to contact the shaft and to extend from the distal side in a proximal direction toward the motor.

17. The heart pump of Embodiment 16, further comprising a proximal radial shaft seal having a proximal side configured to face proximally toward the motor and a radially inner lip configured to contact the shaft and extend from the proximal side in a distal direction toward the impeller.

18. The heart pump of any of Embodiments 16 to 17, further comprising one or more discs within the housing having an opening with an inner diameter that is less than an outer diameter of the shaft.

19. The heart pump of any of Embodiments 16 to 18, further comprising a seal housing, wherein the seal and seal housing are configured to be inserted as an integrated unit over the shaft.

20. The heart pump of any of Embodiments 16 to 19, wherein the heart pump is configured to be delivered to the heart via a catheter.

21. A seal assembly for a heart pump, comprising:
a housing having a distal end wall and a cylindrical side wall, the side wall extending axially and proximally from the distal end wall to define a cavity, the distal end wall having a distal side configured to contact blood flow and having a central opening configured to receive therethrough a shaft having an outer diameter;
a distal disc inside the cavity located proximally of the distal end wall;
a distal radial shaft seal inside the cavity located proximally of the distal disc, with a flat side facing distally and an open side facing proximally;
a proximal radial shaft seal inside the cavity located proximally of the distal radial shaft seal, with a flat side facing proximally and an open side facing distally; and
a middle disc inside the cavity located proximally of the distal radial shaft seal and distally of the proximal radial shaft seal.

22. The seal assembly of Embodiment 21, further comprising a proximal disc located proximally of the proximal radial shaft seal and configured to be spring-loaded when assembled with the heart pump to apply a compressive force in the distal direction on the proximal radial shaft seal.

23. The seal assembly of any of Embodiments 21 to 22, further comprising:
a distal spring located at least partially within the open side of the distal radial shaft seal and configured to compress a radially inner lip of the distal radial shaft seal radially inwardly onto the shaft; and
a proximal spring located at least partially within the open side of the proximal radial shaft seal and configured to compress a radially inner lip of the proximal radial shaft seal radially inwardly onto the shaft.

24. The seal assembly of any of Embodiments 21 to 23, wherein each of the distal disc and the middle disc comprises a central opening with an inner diameter configured to be less than the outer diameter of the shaft.

25. The seal assembly of Embodiment 24, wherein a radially inner edge of the central opening of each of the distal and middle discs is configured to wear off in response to rotation of the shaft.

26. The seal assembly of any of Embodiments 21 to 25, further comprising grease located between the distal radial shaft seal and the middle disc and between the middle disc and the proximal radial shaft seal.

27. The seal assembly of any of Embodiments 21 to 26, wherein each of the distal and proximal radial shaft seals have radially inner lips that contact the shaft.

28. The seal assembly of any of Embodiments 21 to 27, wherein each of the distal and proximal radial shaft seals have radially outer lips that contact the housing.

29. The seal assembly of any of Embodiments 21 to 28, wherein the seal assembly is configured to be inserted as an integrated unit over the shaft and at least partially into a heart pump housing.

30. The seal assembly of any of Embodiments 21 to 29, wherein the seal assembly is configured to be assembled with the heart pump and delivered to the heart via a catheter.

31. The seal assembly of any of Embodiments 21 to 30, wherein the housing is a seal housing configured to be coupled with a motor housing that supports a motor of the heart pump.

32. The seal assembly of any of Embodiments 21 to 30, wherein the housing is a motor housing configured to support a motor of the heart pump.

33. A seal assembly for a heart pump, the heart pump having a motor configured to rotate an impeller via a shaft about an axis, the seal assembly comprising:
a housing; and
a distal radial shaft seal within the housing having a flat side configured to face distally toward the impeller and a radially inner lip configured to contact the shaft and to extend from the flat side in a proximal direction toward the motor.

34. The seal assembly of Embodiment 33, further comprising a proximal radial shaft seal within the housing having a flat side configured to face proximally toward the motor and a radially inner lip configured to extend from the flat side in a distal direction along the shaft toward the impeller.

35. The seal assembly of any of Embodiments 33 to 34, further comprising one or more discs within the housing having an opening with an inner diameter that is less than an outer diameter of the shaft.

36. The seal assembly of any of Embodiments 33 to 35, wherein the seal assembly is configured to be inserted as an integrated unit over the shaft.

37. The seal assembly of any of Embodiments 33 to 36, wherein the distal radial shaft seal is elastomeric.

38. The seal assembly of any of Embodiments 33 to 37, wherein the housing is a seal housing configured to be coupled with a motor housing that supports a motor of the heart pump.

39. The seal assembly of any of Embodiments 33 to 37, wherein the housing is a motor housing configured to support a motor of the heart pump.

40. A heart pump comprising:
an impeller;
a motor configured to rotate the impeller via a shaft about an axis; and
a seal assembly comprising:
a housing; and
a distal radial shaft seal within the housing having a flat side configured to face distally toward the impeller and a radially inner lip configured to contact the shaft and to extend from the flat side in a proximal direction toward the motor.

41. The heart pump of Embodiment 40, further comprising a proximal radial shaft seal within the housing having a flat side configured to face proximally toward the motor and a radially inner lip configured to contact the shaft and to extend from the flat side in a distal direction toward the impeller.

42. The heart pump of any of Embodiments 40 to 41, further comprising one or more discs within the housing having an opening with an inner diameter that is less than an outer diameter of the shaft.

43. The heart pump of any of Embodiments 40 to 42, wherein the seal assembly is configured to be inserted as an integrated unit over the shaft.

44. The heart pump of any of Embodiments 40 to 43, wherein the heart pump is configured to be delivered to the heart via a catheter.

45. The heart pump of any of Embodiments 40 to 44, wherein the housing is a seal housing configured to be coupled with a motor housing that supports the motor.

46. The heart pump of any of Embodiments 40 to 44, wherein the housing is a motor housing configured to support the motor.

47. A heart pump (22) comprising:
a motor (145) having a rotor;
an impeller (72) for providing a blood flow;
a drive shaft (140) that is connected to the rotor and the impeller; and
a seal element (156) that is disposed between the motor and the impeller,
wherein the seal element (156) includes a central aperture for receiving the drive shaft (140) in sealing contact.

48. A heart pump (22) of Embodiment 47, wherein the motor (145) is contained within a motor housing (164), a portion of the drive shaft (140) extends from the motor housing.

49. A heart pump (22) of Embodiment 48, wherein the seal element (156) is disposed between a wall of the motor housing (164) and the drive shaft (140).

50. A heart pump (22) of any preceding Embodiments 47 to 49, wherein the seal element (156) is positioned at least in part in the motor housing.

51. A heart pump (22) of any preceding Embodiments 48 to 50, wherein the seal element (156) is connected to the motor housing (164)

52. A heart pump (22) of any preceding Embodiments 48 to 50, wherein the seal element (156) is connected to the drive shaft (164)

53. A heart pump (22) of any preceding Embodiments 48 to 52, wherein the motor housing has an outer diameter in a range of 4 to 5 mm.

54. A heart pump (22) of any preceding Embodiments 48 to 52, wherein the motor housing has an outer diameter no greater than 5 mm.

55. A heart pump (22) of any preceding Embodiments 48 to 54, wherein the motor housing has a length no greater than 33 mm, optionally no greater 25.5 mm.

56. A heart pump (22) of any preceding Embodiments 48 to 55, wherein the seal element (156) is contained at least partially in a seal housing (240).

57. A heart pump (22) of Embodiment 56, wherein the motor housing (164) is configured to be welded to the seal housing (240).

58. A heart pump (22) of Embodiment 57, wherein the seal housing (240) comprises an outer surface recess (245), the motor housing has an inner surface, and the outer surface recess is mated with the inner surface.

59. A heart pump (22) of Embodiment 57 or 58, wherein the seal housing (240) has an outer surface rabbet (246) and the motor housing has an outer surface rabbet (247), and the seal housing is attached to the motor housing (164) with a weld where the seal housing rabbet meets the motor housing rabbet.

60. A heart pump (22) of any preceding Embodiments 47 to 59, wherein the drive shaft (140), rotor, impeller (72), and seal element (156) each share a central axis.

61. A heart pump (22) of any preceding Embodiments 47 to 59, wherein at least a portion of the drive shaft (140) is flexible.

62. A heart pump (22) of Embodiment 60 or 61, wherein the drive shaft comprises a sleeve 154.

63. A heart pump (22) of any preceding Embodiments 60 to 62, wherein the drive shaft comprises a surface treatment, optionally comprising electropolishing, nitriding, a hydrophilic coating (optionally Polyvinylpyrrolidon having a thickness in a range of 3 to 5 μm), a hydrophobic coating (optionally Perfluoralkoxy having a thickness in a range of 10 to 20 μm), or a micropatterned surface.

64. A heart pump (22) of any preceding Embodiments 60 to 63, wherein the drive shaft (140) has a length in a range of 1200 mm to 1500 mm.

65. A heart pump (22) of Embodiment 64, wherein the drive shaft (140) has a length in a range of 29 to 34 mm.

66. A heart pump (22) of any preceding Embodiments 47 to 65, wherein the impeller (72) is connected to the drive shaft (140) at a proximal end of the impeller.

67. A heart pump (22) of Embodiment 66, wherein the distal end of the impeller is freely floating.

68. A heart pump (22) of Embodiment 66 or 67, wherein the impeller (72) comprises a central hub (146), the central hub comprises a central bore (226), and the drive shaft (140) is positioned in the central bore.

69. A heart pump (22) of Embodiment 68, wherein the impeller (72) further comprises at least one side bore (227) in communication with the central bore (226).

70. A heart pump (22) of Embodiment 69, wherein the side bore (227) is distal to the drive shaft (140).

71. A heart pump (22) of any preceding Embodiments 68 to 70, wherein an impeller base plate (152) is connected to the drive shaft (140) and the impeller (72).

72. A heart pump (22) of Embodiment 71, wherein at least a portion of the impeller base plate (152) is positioned between the drive shaft (140) and the central bore (226).

73. A heart pump (22) of Embodiment 71, wherein the impeller base plate (152) comprises a tubular extension (154) that is part of the drive shaft.

74. A heart pump (22) of any preceding Embodiments 47 to 73, wherein the impeller has a base flange (150).

75. A heart pump (22) of Embodiment 74 in combination with Embodiment 68, wherein the central hub (146) transitions to the base flange (150) with a smooth concave curve or taper.

76. A heart pump (22) of Embodiment 74 in combination with Embodiment 48, wherein the base flange has a diameter that is 0 mm to 0.1 mm less than an outer diameter of the motor housing (164).

77. A heart pump (22) of any preceding Embodiments 47 to 76, wherein the impeller (72) comprises radial flow blades (177) arranged on a plane perpendicular to an axis of rotation of the impeller.

78. A heart pump (22) of Embodiment 77 in combination with Embodiment 74, wherein the radial flow blades (177) are on a proximal surface of the impeller base flange (150).

79. A heart pump (22) of Embodiment 77 in combination with Embodiment 71, wherein the radial flow blades (177) are on a proximal surface of the impeller base plate (152).

80. A heart pump (22) of any preceding Embodiments 77 to 79, wherein the radial flow blades (177) are protrusions or indentations extending radially from an axis of rotation of the impeller (72).

81. A heart pump (22) of any preceding Embodiments 77 to 80, wherein the radial flow blades (177) are one of straight or curved.

82. A heart pump (22) of any preceding Embodiments 77 to 81, wherein the seal element (156) and the radial flow blades (177) are separated by an axial gap (174), and wherein the axial gap has a distance in a range of 0.08 mm to 0.3 mm.

83. A heart pump (22) of any preceding Embodiments 77 to 82, wherein the radial flow blades (177) are arranged to be radially symmetric about an axis of rotation of the impeller (72).

84. A heart pump (22) of any preceding Embodiments 77 to 83, wherein the radial flow blades (177) comprise a surface treatment, optionally comprising electropolishing, nitriding, a hydrophilic coating (optionally Polyvinylpyrrolidon having a thickness in a range of 3 to 5 μm), a hydrophobic coating (optionally Perfluoralkoxy having a thickness in a range of 10 to 20 μm), or a micropatterned surface.

85. A heart pump (22) of any preceding Embodiments 47 to 84, wherein the seal element (156) comprises a rotary shaft lip seal having a seal holder (166), an elastomeric annular seal (167), a seal cavity (176), and a garter spring (168) positioned in the seal cavity, and wherein the elastomeric annular seal (167) comprises a contact lip (169).

86. A heart pump (22) of Embodiment 85 in combination with Embodiment 2, wherein the seal holder (166) is configured to remain stationary with respect to the motor housing (164), and the contact lip (169) is configured to be in contact with the drive shaft (140).

87. A heart pump (22) of Embodiment 85 in combination with Embodiment 2, wherein the seal holder (166) is configured to remain stationary with respect to the drive shaft (140), and the contact lip (169) is configured to be in contact with the motor housing (164).

88. A heart pump (22) of any preceding Embodiments 85 to 87, wherein the seal cavity (176) is defined at least in part by the seal holder (166) and the elastomeric annular seal (167),

89. A heart pump (22) of any preceding Embodiments 85 to 88, wherein a first grease (175) is disposed in the seal cavity (176).

90. A heart pump (22) of any preceding Embodiments 85 to 89, wherein the seal cavity (176) is oriented distally.

91. A heart pump (22) of any preceding Embodiments 85 to 89, wherein the seal cavity (176) is oriented proximally.

92. A heart pump (22) of any preceding Embodiments 85 to 91, further comprising a distal disc (255) located distal to the seal element (156).

93. A heart pump (22) of Embodiment 92, wherein the distal disc (255) has a distal surface configured to contact blood when in use.

94. A heart pump (22) of any preceding Embodiments 92 to 93, wherein the distal disc (255) is made at least in part from stainless steel, titanium, PTFE, PEEK, or polyurethane.

95. A heart pump (22) of any preceding Embodiments 92 to 94, wherein the distal disc (255) comprises a central opening with an inner diameter that is greater than the outer diameter of the drive shaft (140), optionally by a difference in a range of 0.02 to 0.1 mm.

96. A heart pump (22) of any preceding Embodiments 92 to 94, wherein the distal disc (255) comprises a central opening with an inner diameter that is less than the outer diameter of the drive shaft (140), optionally by a difference in a range of 0.02 to 0.1 mm.

97. A heart pump (22) of any preceding Embodiments 92 to 96, wherein the distal disc (255) has a thickness (172) that is uniform and in a range of 0.1 mm to 1.5 mm, optionally about 1.0 mm.

98. A heart pump (22) of any preceding Embodiments 92 to 97, in combination with Embodiment 48, wherein the distal disc (255) comprises a form-fitting feature (173) configured to provide a tight connection to the motor housing (164).

99. A heart pump (22) of any preceding Embodiments 92 to 98, wherein at least a distal surface of the distal disc (255) comprises a surface treatment, optionally comprising electropolishing, nitriding, a hydrophilic coating (optionally Polyvinylpyrrolidon having a thickness in a range of 3 to 5 μm), a hydrophobic coating (optionally Perfluoralkoxy having a thickness in a range of 10 to 20 μm), or a micropatterned surface.

100. A heart pump (22) of any preceding Embodiments 85 to 99, further comprising a proximal disc (275) located adjacent and proximal to the seal element (156).

101. A heart pump (22) of Embodiment 100, wherein the proximal disc (275) is made at least in part from stainless steel, titanium, PTFE, PEEK, or polyurethane.

102. A heart pump (22) of any preceding Embodiments 100 to 101, wherein the proximal disc (275) comprises a central opening with an inner diameter that is greater than the outer diameter of the drive shaft (140), optionally by a difference in a range of 0.02 to 0.1 mm.

103. A heart pump (22) of any preceding Embodiments 100 to 101, wherein the proximal disc (275) comprises a central opening with an inner diameter that is less than the outer diameter of the drive shaft (140), optionally by a difference in a range of 0.02 to 0.1 mm.

104. A heart pump (22) of any preceding Embodiments 100 to 103, wherein the proximal disc (275) has a thickness that is uniform and in a range of 0.1 mm to 1.5 mm, optionally about 1.0 mm.

105. A heart pump (22) of any preceding Embodiments 100 to 104, in combination with Embodiment 48, wherein the proximal disc (275) comprises a form-fitting feature configured to provide a tight connection to the motor housing (164).

106. A heart pump (22) of any preceding Embodiments 100 to 105, wherein the proximal disc is axially spring-loaded.

107. A heart pump (22) of any preceding Embodiments 100 to 105, wherein a proximal cavity (189) is defined at least in part by the proximal disc (275) and the seal element (156) and wherein a second grease is located in the proximal cavity (189).

108. A heart pump (22) of Embodiment 107, wherein the second grease has a lower consistency than the first grease.

109. A heart pump (22) of Embodiment 90 or 91, further comprising a distal protection disc (212), wherein the distal protection disc (212) comprises a central opening (213) and a distally facing conical surface (214) with a concave contour.

110. A heart pump (22) of Embodiment 109, wherein at least a portion of the central opening (213) has a diameter that is greater than the outer diameter of the drive shaft (140), optionally by a difference in a range of 0.08 to 0.15 mm.

111. A heart pump (22) of Embodiment 110, wherein at least a portion of the central opening (213) has a diameter that is less than the outer diameter of the drive shaft (140), optionally by a difference in a range of 0.01 to 0.05 mm.

112. A heart pump (22) of any preceding Embodiments 109 to 111 in combination with Embodiment 2, wherein the distal protection disc (212), is connected to the motor housing (164).

113. A heart pump (22) of any preceding Embodiments 109 to 112, wherein the distal protection disc (212) is made from an elastomeric material, optionally PTFE or PEEK.

114. A heart pump (22) of any preceding Embodiments 109 to 113 in combination with Embodiment 21, wherein the conical surface (214) aligns with a surface of the hub (146).

115. A heart pump (22) of any preceding Embodiments 109 to 114 in combination with Embodiment 21, wherein the distal protection disc (212) comprises a flat surface portion (215) adjacent to the conical surface (214), the hub (146) has a flat base, and the flat surface portion (215) has a diameter within 0.01 mm of a diameter of the flat base.

116. A heart pump (22) of any preceding Embodiments 109 to 115, wherein the impeller (72) comprises an overlap impeller (210) comprising at least two impeller blades (178), wherein the at least two impeller blades (178) each comprise a proximal portion (211) shaped to follow the conical surface (214).

117. A heart pump (22) of any preceding Embodiments 109 to 115, wherein the impeller (72) comprises at least two impeller blades (178), wherein the at least two impeller blades (178) each comprise a proximal portion (211) with a flat edge (225).

118. A heart pump (22) of any preceding Embodiments 85 to 117, wherein the seal element (156) further comprises a second rotary shaft lip seal having a second seal holder (166b), a second elastomeric annular seal (167b), and a second seal cavity (176b), and wherein the second elastomeric annular seal (167b) comprises a second contact lip (169b).

119. A heart pump (22) of Embodiment 118, further comprising a second garter spring (168b) positioned in the second seal cavity (176b).

120. A heart pump (22) of any preceding Embodiments 118 to 119, wherein the second seal cavity (176b) is oriented toward the first seal cavity (176, 176a).

121. A heart pump (22) of any preceding Embodiments 118 to 120, wherein the contact lip (169, 169a) and the second contact lip (169b) are oriented toward one another.

122. A heart pump (22) of any preceding Embodiments 118 to 121 in combination with Embodiment 89, wherein the first grease is disposed in the second seal cavity (176b).

123. A heart pump (22) of any preceding Embodiments 118 to 121 in combination with Embodiment 89, wherein a third grease is disposed in the second seal cavity (176b), the third grease having different properties than the first grease.

124. A heart pump (22) of any preceding Embodiments 118 to 123, further comprising a middle disc (260) located axially between the rotary shaft lip seal and the second rotary shaft lip seal.

125. A heart pump (22) of Embodiment 124, wherein the middle disc (260) is made from an elastomeric material, optionally PTFE or PEEK.

126. A heart pump (22) of any preceding Embodiments 124 to 125, wherein the middle disc (260) has a central opening, and at least a portion of the central opening has a diameter (261) that is less than the outer diameter of the drive shaft (140), optionally by a difference in a range of 0.01 to 0.05 mm.

127. A heart pump (22) of any preceding Embodiments 124 to 125 in combination with Embodiment 123, wherein the third grease and the first grease are separated by the middle disc (260).

128. A heart pump (22) of any preceding Embodiments 118 to 127, wherein at least one of the elastomeric annular seal (167, 167a) and the second elastomeric annular seal (167b) comprises a leading edge, wherein the leading edge has a central hole that is less than an outer diameter of the drive shaft (140).

129. A heart pump (22) of Embodiment 128, wherein the leading edge central hole is in a range of 80% to 90% of the outer diameter of the drive shaft (140).

130. A heart pump (22) of any preceding Embodiments 128 to 129, wherein the leading edge is located distal to the contact lip (169) and the second contact lip (169b).

131. A heart pump (22) of any preceding Embodiments 47 to 130, further comprising an axial face lip seal (300) positioned proximal to the impeller (72), the axial face lip seal (300) comprising a lip configured to slidably contact a proximal end of the impeller.

132 A heart pump (22) of Embodiment 131 in combination with Embodiment 85, wherein a fluid barrier reservoir is defined by the axial face lip seal (300), a base of the impeller (72), and the rotary shaft lip seal, and wherein a fluid is deposited in the fluid barrier reservoir.

133. A heart pump (22) of any preceding Embodiments 47 to 132, wherein the seal element (156) is contained at least partially within a seal housing (240).

134. A heart pump (22) of Embodiment 133, wherein the seal housing (240) comprises a distal end wall and a cylindrical side wall, the side wall extending axially and proximally from the distal end wall to define a cavity (248), the distal end wall having a distal side (241) configured to contact blood flow and having a central opening (242) configured to receive therethrough a shaft (140) having an outer diameter (141);

135. A heart pump (22) of Embodiment 134, wherein the distal side (241) is a smooth flat surface.

136. A heart pump (22) of any preceding Embodiments 134 to 135, wherein the distal side (241) comprises a surface treatment, optionally comprising electropolishing, nitriding, a hydrophilic coating (optionally Polyvinylpyrrolidon having a thickness in a range of 3 to 5 μm), a hydrophobic coating (optionally Perfluoralkoxy having a thickness in a range of 10 to 20 μm), or a micropatterned surface.

137. A heart pump (22) of any preceding Embodiments 133 to 136, further comprising a seal container cap (278) configured to connect to the seal housing (240) and contain the seal element (156).

138 A heart pump (22) of Embodiment 137, wherein the seal housing (240) and the seal container cap (278) both have central openings having inner diameters greater than an outer diameter (141) of the drive shaft (140), optionally by a difference in a range of 0.08 to 0.15 mm.

139. A heart pump (22) of any preceding Embodiments 133 to 138 in combination with Embodiment 2, wherein the seal housing (240) is configured to be at least partially inserted into the motor housing (164).

140. A heart pump (22) of any preceding Embodiments 47 to 139, further comprising a pressure balancing element in fluid communication with the seal element (156), the pressure balancing element being responsive to changes in blood pressure in the patient's heart when in use.

141. A heart pump (22) of Embodiment 140 in combination with Embodiment 85, wherein the pressure balancing element comprises:
a channel between the seal cavity (176, 176a) and an environment external to the heart pump (22); and
a diaphragm covering the channel.

142. A heart pump (22) of any preceding Embodiments 140 to 141, wherein the pressure balancing element further comprises a lubricant reservoir (290).

143. A heart pump (22) of Embodiment 142 in combination with Embodiment 2, wherein the lubricant reservoir (290) is a recess in the motor housing (164).

144. A heart pump (22) of Embodiment 142 or 143 in combination with Embodiment 2, wherein the lubricant reservoir (290) is an annular groove in an inner surface of the motor housing (164).

145. A heart pump (22) of any preceding Embodiments 140 to 144, wherein a lubricant is deposited in the pressure balancing element.

146. A heart pump (22) of any preceding Embodiments 142 to 145, wherein a lubricant is deposited in the lubricant reservoir (290).

147. A heart pump (22) of Embodiment 145 or 146, wherein the lubricant has a viscosity in a range of 0.30 to 1.30 mPa·s.

148. A heart pump (22) of any preceding Embodiments 141 to 147, wherein the channel comprises at least one of a seal holder channel (291), a seal housing channel, a motor housing channel (294), or an inlet tube channel (293).

149. A heart pump (22) of any preceding Embodiments 141 to 148 in combination with Embodiment 118, wherein the channel is in fluid communication with the second seal cavity (176b).

150. A heart pump (22) of any preceding Embodiments 141 to 149 in combination with Embodiment 35, wherein the environment external to the heart pump is within 20 mm of the axial gap (174).

151. A heart pump (22) of any preceding Embodiments 141 to 150, wherein the environment external to the heart pump is in the patient's left ventricle.

152. A heart pump (22) of any preceding Embodiments 141 to 151, wherein the diaphragm (292) is made from silicone.

153. A heart pump (22) of any preceding Embodiments 141 to 152, wherein the diaphragm (292) is positioned at the radially outer portion of the channel.

154. A heart pump (22) of any preceding Embodiments 141 to 153, comprising an inlet tube (70) having a pressure balancing port (293) having a diameter that is smaller than a diameter of the diaphragm (202), and wherein the diaphragm (292) is positioned radially under the pert (293).

155. A heart pump (22) of any preceding Embodiments 141 to 154, wherein the channel comprises a plurality of radially extending channels.

156. A heart pump (22) of any preceding Embodiments 47 to 155, further comprising a superabsorber located within the seal element (156).

157. A heart pump (22) of Embodiment 156, wherein the superabsorber is carried on a piece of foil or cellulose.

158. A heart pump (22) of any preceding Embodiments 156 to 157, wherein the superabsorber is positioned in at least one of a seal cavity (176a), a second seal cavity (176b), on a distal disc (255), a middle disc (260), a proximal disc (275), a grease (175), a second grease, or a third grease.

159. A heart pump (22) of any preceding Embodiments 156 to 158, wherein the superabsorber comprises sodium polyacrylate.

160. A heart pump (22) of any preceding Embodiments 47 to 159, wherein the impeller (72) is connected to an impeller base plate (152) and the impeller base plate (152) is connected to the drive shaft (140).

161. A heart pump (22) of Embodiment 160, wherein the impeller base plate (152) comprises a tubular extension (154).

162. A heart pump (22) of Embodiment 161, wherein the tubular extension (154) is positioned at least partially in a central bore (226) of the impeller (72).

163. A heart pump (22) of Embodiment 161, wherein the tubular extension (154) is positioned at least partially in the seal element (156).

164. A heart pump (22) of any preceding Embodiments 160 to 163, wherein the impeller (72) comprises a recess (311), the impeller base plate (152) comprises a recess (310), and key (309) is positioned in the recess (311) and the recess (310).

165. A heart pump (22) of Embodiment 164, wherein the key (309) is welded to the drive shaft (140).

166. A heart pump (22) of any preceding Embodiments 164 to 165, wherein the key (309) is non-circular in a plant transverse to an axis of rotation of the impeller (72).

167. A heart pump (22) of any preceding Embodiments 160 to 166, wherein the impeller (72) is welded to the impeller base plate (152).

168. A heart pump (22) of any preceding Embodiments 160 to 166, wherein the impeller (72) and the impeller base plate (152) are different materials.

169. A heart pump (22) of any preceding Embodiments 47 to 168, wherein the seal element is configured to maintain functionality for at least 12 hours.

170. A heart pump (22) of any preceding Embodiments 47 to 168, wherein the seal element is configured to lose functionality due to wear after 12 hours.

171. A controller for providing power to a motor of a heart pump comprising a control algorithm, wherein the motor is a field-oriented control motor, and the control algorithm adjusts the power based on a feedback signal from the field-oriented control motor to maintain the motor within a rotational speed set point range.

172. A controller of Embodiment 171, wherein the rotational speed set point range is a rotational speed plus or minus 1%.

173. A system comprising the heart pump (22) of any preceding Embodiments 47 to 168 and a controller of Embodiment 171 or 172.

174. A heart pump (22), wherein the heart pump (22) has the following features:
a housing (164) with an interior (302) and an opening (303) to the interior (302);
an impeller (72) with at least one blade (178), wherein the impeller (72) is located proximate to the opening (303);
a motor (115) disposed in the interior (302) and having a shaft (140) passing through the opening (303) and coupled to the impeller (72) for driving the impeller (72);
a sealing element (300) that is arranged between the impeller (72) and the housing (164) and is adapted to seal a gap 174 between the impeller (72) and the housing (164); and
a barrier fluid (301) disposed between the seal member (300) and the shaft (140) and adapted to prevent ingress of a medium from an environment of the heart pump (22) into an interior of the motor (115).

175. A heart pump (22) according to Embodiment 174, wherein the sealing element (300) is attached to the impeller (72).

176. A heart pump (22) according to Embodiment 174, wherein the sealing element (300) is attached to the housing (164).

177. A heart pump (22) according to any of the preceding Embodiments, wherein the barrier fluid (301) is further contained in the interior of the motor (115).

178. A heart pump (22) according to one of the preceding Embodiments, wherein the sealing element (300) is formed as a contact or non-contact seal.

179. A heart pump (22) according to one of the preceding Embodiments, wherein the sealing element (300) is designed as a labyrinth seal and/or gap seal.

180. A heart pump (22) according to any one of the preceding Embodiments, with a further sealing element (167), wherein the further sealing element (167) is arranged at the opening (303) and is formed to seal the interior space (302) of the housing (164) against a fluid located between the housing (164) and the impeller (72), wherein the barrier fluid (301) is arranged in the space (305).

181. A heart pump (22) according to any one of the preceding Embodiments, with at least one bearing (162), wherein the bearing (162) is designed to store the shaft (140) against the housing (164).

182. A heart pump (22) according to any one of the preceding Embodiments, wherein the barrier fluid (301) is a biocompatible medium.

183. A heart pump (22) according to any one of the preceding Embodiments, wherein the barrier fluid (301) consists of glucose and/or endogenous fat.

184. A heart pump (22) according to any one of the preceding Embodiments, and in combination with Embodiment 100, wherein the proximal sealing disc (275) comprises an axial face seal on its proximal side slidably engaged with a bearing.

185. A heart pump (22) according to any one of the preceding Embodiments, and in combination with Embodiment 56, wherein the seal housing (240) has a distally facing conical surface 321.

186. A heart pump (22) according to Embodiment 185, wherein the distal disc (255) comprises a tubular extension (322) having a radially inward surface, wherein at least a portion of the radially inward surface contacts the motor shaft (140).

187. A heart pump (22) according to Embodiment 186, wherein the tubular extension (322) has a surface texture or treatment on the radially inward surface, the surface texture or treatment optionally comprising circumferential ribs, indents, a hydrophilic micropattern, or a hydrophobic micropattern.

188. A heart pump (22) according to Embodiment 186, wherein the tubular extension is made from a biocompatible elastomer.

189. A heart pump (22) according to Embodiment 186, wherein the tubular extension is made from an elastomer or thermoplastic and a cavity is adjacent to the tubular extension optionally in the seal housing (240), the cavity configured to hold a lubricant and deliver the lubricant to the tubular extension.

190. A heart pump (22) according to any of Embodiments 184 to 189, wherein the tubular extension has a distal surface (323) that is flush with a distal end of the seal housing (240).

191. A heart pump (22) according to any of Embodiments 184 to 189, wherein the tubular extension has a distal surface (323) that extends distally beyond the seal housing (240), optionally in a range of 0 to 200 microns, preferably about 100 microns.

192. A heart pump (22) according to Embodiment 191, wherein the distal surface (323) is an axial face seal that slidably contacts the impeller (72).

193. A heart pump (22) according to any Embodiments 185 to 192, further comprising outlet strut supports (325).

194. A heart pump (22) according to Embodiment 193, wherein each of the outlet strut supports connect to an outlet strut (195) at a position at least between a proximal end and a distal end of the outlet strut (195).

195. A heart pump (22) according to Embodiment 194, wherein the outlet strut supports (325) are connected to or part of the seal housing (240), optionally the distally facing conical face (321).

196. A heart pump (22) according to Embodiment 195, wherein the outlet strut supports (325) comprise an axial length (326) that is a portion of the outlet strut length, optionally the portion is up to 30%, up to 50%, or up to 100% of the outlet strut length.

197. A heart pump (22) according to Embodiment 195, wherein the outlet strut supports (325) comprise an axial length (326) that is a portion of the axial length of the conical face (321), optionally the portion is up to 30%, up to 50%, or up to 100% of the axial length of the conical face.

198. A heart pump (22) according to any Embodiment 193 to 197, wherein the outlet strut supports (325) comprise a rounded leading edge (328).

199. A heart pump (22) according to Embodiment 198, wherein the outlet strut supports (325) comprise a surface angled from the leading edge (328) to an adjacent outlet window (68).

200. A heart pump (22) according to Embodiment 198 or 199, wherein the leading edge (328) is centered within a width of the connecting outlet strut (195).

201. A heart pump (22) according to Embodiment 198 or 199, wherein the leading edge (328) is positioned within a width of the connecting outlet strut (195) and near or adjacent an edge of the connecting outlet strut (195) on a side facing a radial component of blood flow.

202. A heart pump (22) according to any one of the preceding Embodiments, and in combination with Embodiment 100, wherein the proximal sealing disc (275) comprises a first thickness (282) and a second thickness (283) that is thicker and closer to the central axis (185) than the first thickness (282).

203. A heart pump (22) according to Embodiment 202, in combination with Embodiment 137 wherein the second thickness (283) is greater than a combination of the first thickness (282) and a thickness of the seal container cap (278).

What is claimed is:

1. A seal for a heart pump, the seal comprising:
a distal radial shaft seal configured to surround a motor shaft of the heart pump, with a flat side of the distal radial shaft seal facing distally and an open side of the distal radial shaft seal facing proximally; and
a proximal radial shaft seal configured to surround the shaft and be located proximally of the distal radial shaft seal, such that the proximal radial shaft seal is located farther from an impeller of the pump than the distal radial shaft seal, with a flat side of the proximal radial shaft seal facing proximally and an open side of the proximal radial shaft seal facing distally.

2. The seal of claim 1, wherein the distal radial shaft seal comprises a radially inner lip configured to contact the shaft and to extend from the flat side of the distal radial shaft seal in a proximal direction.

3. The seal of claim 1, further comprising a distal spring located at least partially within the open side of the distal radial shaft seal and configured to compress a radially inner lip of the distal radial shaft seal radially inwardly onto the shaft.

4. The seal of claim 3, further comprising a proximal spring located at least partially within the open side of the proximal radial shaft seal and configured to compress a radially inner lip of the proximal radial shaft seal radially inwardly onto the shaft.

5. The seal of claim 1, further comprising one or more discs comprising a central opening with an inner diameter configured to be less than the outer diameter of the shaft.

6. The seal of claim 5, wherein a radially inner edge of the central opening of each of the discs is configured to wear off in response to rotation of the shaft.

7. The seal of claim 6, further comprising grease located between the distal radial shaft seal and a middle disc and between the middle disc and the proximal radial shaft seal.

8. The seal of any of claims 1 to 7, wherein the seal is configured to be assembled with the heart pump and delivered to the heart via a catheter.

9. The seal of any of claims 1 to 7, further comprising a housing having a distal end wall and a cylindrical side wall extending proximally from the distal end wall, the distal end wall having a distal side configured to contact blood flow and having a central opening configured to receive therethrough the shaft, wherein the distal radial shaft seal is configured to be located proximally of the distal end wall at least partially within the housing.

10. A seal assembly for a heart pump, comprising:
a housing having a distal end wall and a cylindrical side wall, the side wall extending axially and proximally from the distal end wall to define a cavity, the distal end wall having a distal side configured to contact blood flow and having a central opening configured to receive therethrough a shaft having an outer diameter;
a distal disc inside the cavity located proximally of the distal end wall;
a distal radial shaft seal inside the cavity located proximally of the distal disc, with a flat side facing distally and an open side facing proximally;
a proximal radial shaft seal inside the cavity located proximally of the distal radial shaft seal, with a flat side facing proximally and an open side facing distally; and
a middle disc inside the cavity located proximally of the distal radial shaft seal and distally of the proximal radial shaft seal.

11. The seal assembly of claim 10, further comprising a proximal disc located proximally of the proximal radial shaft seal and configured to be spring-loaded when assembled with the heart pump to apply a compressive force in the distal direction on the proximal radial shaft seal.

12. The seal assembly of any of claims 10 to 11, further comprising:
a distal spring located at least partially within the open side of the distal radial shaft seal and configured to compress a radially inner lip of the distal radial shaft seal radially inwardly onto the shaft; and
a proximal spring located at least partially within the open side of the proximal radial shaft seal and configured to compress a radially inner lip of the proximal radial shaft seal radially inwardly onto the shaft.

13. The seal assembly of any of claims 10 to 11, wherein each of the distal disc and the middle disc comprises a central opening with an inner diameter configured to be less than the outer diameter of the shaft.

14. The seal assembly of claim 13, wherein a radially inner edge of the central opening of each of the distal and middle discs is configured to wear off in response to rotation of the shaft.

15. The seal assembly of any of claims 10 to 11, further comprising grease located between the distal radial shaft seal and the middle disc and between the middle disc and the proximal radial shaft seal.

16. The seal assembly of any of claims 10 to 11, wherein each of the distal and proximal radial shaft seals have radially inner lips that contact the shaft.

17. The seal assembly of any of claims 10 to 11, wherein each of the distal and proximal radial shaft seals have radially outer lips that contact the housing.

18. The seal assembly of any of claims 10 to 11, wherein the seal assembly is configured to be inserted as an integrated unit over the shaft and at least partially into a heart pump housing.

19. The seal assembly of any of claims 10 to 11, wherein the seal assembly is configured to be assembled with the heart pump and delivered to the heart via a catheter.

20. The seal assembly of any of claims 10 to 11, wherein the housing is a seal housing configured to be coupled with a motor housing that supports a motor of the heart pump.

21. The seal assembly of any of claims 10 to 11, wherein the housing is a motor housing configured to support a motor of the heart pump.

22. A heart pump (22) comprising:
a motor (145) having a rotor;
an impeller (72) for providing a blood flow;
a drive shaft (140) that is connected to the rotor and the impeller; and
a seal element (156) that is disposed between the motor and the impeller,
wherein the seal element (156) includes a central aperture for receiving the drive shaft (140) in sealing contact.

23. The heart pump (22) of claim 22, wherein the motor (145) is contained within a motor housing (164), a portion of the drive shaft (140) extends from the motor housing.

24. The heart pump (22) of claim 23, wherein the seal element (156) is disposed between a wall of the motor housing (164) and the drive shaft (140).

25. The heart pump (22) of any preceding claims 22 to 24, wherein the seal element (156) is positioned at least in part in the motor housing.

26. The heart pump (22) of any preceding claims 22 to 24, wherein the seal element (156) is connected to the motor housing (164)

27. The heart pump (22) of any preceding claims 23 to 24, wherein the motor housing has an outer diameter no greater than 5 mm.

28. The heart pump (22) of any preceding claims 23 to 24, wherein the motor housing has a length no greater than 33 mm, optionally no greater 25.5 mm.

29. The heart pump (22) of any preceding claims 23 to 24, wherein the seal element (156) is contained at least partially in a seal housing (240).

30. The heart pump (22) of claim 29, wherein the seal housing (240) comprises an outer surface recess (245), the motor housing has an inner surface, and the outer surface recess is mated with the inner surface.

31. The heart pump (22) of claim 30, wherein the seal housing (240) has an outer surface rabbet (246) and the motor housing has an outer surface rabbet (247), and the seal housing is attached to the motor housing (164) with a weld where the seal housing rabbet meets the motor housing rabbet.

32. The heart pump (22) of claim 31, wherein the drive shaft (140) has a length in a range of 29 to 34 mm.

33. The heart pump (22) of claim 32, wherein the impeller (72) is connected to the drive shaft (140) at a proximal end of the impeller.

34. The heart pump (22) of claim 33, wherein the distal end of the impeller is freely floating.

35. The heart pump (22) of claim 33, wherein the impeller (72) comprises a central hub (146), the central hub comprises a central bore (226), and the drive shaft (140) is positioned in the central bore.

36. The heart pump (22) of claim 35, wherein the impeller (72) further comprises at least one side bore (227) in communication with the central bore (226).

37. The heart pump (22) of claim 36, wherein the side bore (227) is distal to the drive shaft (140).

38. The heart pump (22) of claim 35, wherein an impeller base plate (152) is connected to the drive shaft (140) and the impeller (72).

39. The heart pump (22) of claim 22, wherein the impeller (72) comprises radial flow blades (177) arranged on a plane perpendicular to an axis of rotation of the impeller.

40. The pump (22) of claim 39 wherein the impeller has a base flange (150), and, wherein the radial flow blades (177) are on a proximal surface of the impeller base flange (150).

41. The heart pump (22) of claim 39 wherein an impeller base plate (152) is connected to the drive shaft (140) and the impeller (72), and, wherein the radial flow blades (177) are on a proximal surface of the impeller base plate (152).

42. The heart pump (22) of any of claims 39 to 41, wherein the radial flow blades (177) are protrusions or indentations extending radially from an axis of rotation of the impeller (72).

43. The heart pump (22) of any of claims 39 to 42, wherein the radial flow blades (177) are one of straight or curved.

44. The heart pump (22) of any of claims 39 to 43, wherein the seal element (156) and the radial flow blades (177) are separated by an axial gap (174), and wherein the axial gap has a distance in a range of 0.08 mm to 0.3 mm.

45. The heart pump (22) of any of claims 39 to 44, wherein the radial flow blades (177) are arranged to be radially symmetric about an axis of rotation of the impeller (72).

46. The heart pump (22) of claim 22, wherein the seal element is configured to maintain functionality for at least 12 hours.

47. The heart pump (22) of claim 22, wherein the seal element is configured to lose functionality due to wear after 12 hours.
